SEQUENCE LISTING (48 sequences)

SEQ ID NO: 1 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 2 - DNA, 2763 bp, Homo sapiens
  source 1..2763: mol_type = other DNA
atggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 3 - protein, 945 aa, Homo sapiens
MEVQLGLGRVYPRPPSKTYRGAFQNLFQSVREVIQNPGPRHPEAASAAPPGASLLLLQQQQQQQQQQQQQQQQQQQQQQQQQQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 4 - DNA, 2838 bp, Homo sapiens
  source 1..2838: mol_type = other DNA
atggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 5 - RNA, 111 nt, synthetic construct
  source 1..111: mol_type = other RNA
ctggaggcttgctttgggctgtatgctggaatcttccacctacttccctttttggcctctgactgaagggagtattggaagattccaggacacaaggcccttatcagcact

SEQ ID NO: 6 - RNA, 111 nt, synthetic construct
  source 1..111: mol_type = other RNA
ctggaggcttgctttgggctgtatgctgttataacaggcagaagacatcttttggcctctgactgagatgtctcttctgttataacaggacacaaggcccttatcagcact

SEQ ID NO: 7 - RNA, 111 nt, synthetic construct
  source 1..111: mol_type = other RNA
ctggaggcttgctttgggctgtatgctgtttaaaggcatacaacaccatttttggcctctgactgaatggtgtgttgcctttaaacaggacacaaggcccttatcagcact

SEQ ID NO: 8 - RNA, 111 nt, synthetic construct
  source 1..111: mol_type = other RNA
ctggaggcttgctttgggctgtatgctgaatacttgaagggtagattcattttggcctctgactgatgaatctactttcaatattcaggacacaaggcccttatcagcact

SEQ ID NO: 9 - RNA, 208 nt, Homo sapiens
  source 1..208: mol_type = mRNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 10 - skipped

SEQ ID NO: 11 - RNA, 16 nt, Homo sapiens
  source 1..16: mol_type = mRNA
ctctctctctgcaggg

SEQ ID NO: 12 - RNA, 181 nt, Homo sapiens
  source 1..181: mol_type = other RNA
tatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 13 - RNA, 21 nt, synthetic construct
  source 1..21: mol_type = other RNA
agggaagtaggtggaagattc

SEQ ID NO: 14 - RNA, 21 nt, synthetic construct
  source 1..21: mol_type = other RNA
gaatcttccacctacttccct

SEQ ID NO: 15 - RNA, 21 nt, synthetic construct
  source 1..21: mol_type = other RNA
gatgtcttctgcctgttataa

SEQ ID NO: 16 - RNA, 21 nt, synthetic construct
  source 1..21: mol_type = other RNA
ttataacaggcagaagacatc

SEQ ID NO: 17 - RNA, 21 nt, synthetic construct
  source 1..21: mol_type = other RNA
atggtgttgtatgcctttaaa

SEQ ID NO: 18 - RNA, 21 nt, synthetic construct
  source 1..21: mol_type = other RNA
tttaaaggcatacaacaccat

SEQ ID NO: 19 - RNA, 21 nt, synthetic construct
  source 1..21: mol_type = other RNA
tgaatctacccttcaagtatt

SEQ ID NO: 20 - RNA, 21 nt, synthetic construct
  source 1..21: mol_type = other RNA
aatacttgaagggtagattca

SEQ ID NO: 21 - RNA, 21 nt, synthetic construct
  source 1..21: mol_type = other RNA
cattgcaaagaaggctcttag

SEQ ID NO: 22 - RNA, 21 nt, synthetic construct
  source 1..21: mol_type = other RNA
ctaagagccttctttgcaatg

SEQ ID NO: 23 - RNA, 21 nt, synthetic construct
  source 1..21: mol_type = other RNA
cttcttctgcacgagactttg

SEQ ID NO: 24 - RNA, 21 nt, synthetic construct
  source 1..21: mol_type = other RNA
caaagtctcgtgcagaagaag

SEQ ID NO: 25 - RNA, 21 nt, synthetic construct
  source 1..21: mol_type = other RNA
ccctccacgggagactttatt

SEQ ID NO: 26 - RNA, 21 nt, synthetic construct
  source 1..21: mol_type = other RNA
aataaagtctcccgtggaggg

SEQ ID NO: 27 - RNA, 21 nt, synthetic construct
  source 1..21: mol_type = other RNA
actgtggagcaattcattata

SEQ ID NO: 28 - RNA, 21 nt, synthetic construct
  source 1..21: mol_type = other RNA
tataatgaattgctccacagt

SEQ ID NO: 29 - RNA, 21 nt, synthetic construct
  source 1..21: mol_type = other RNA
agaaggagagcagagatttaa

SEQ ID NO: 30 - RNA, 21 nt, synthetic construct
  source 1..21: mol_type = other RNA
ttaaatctctgctctccttct

SEQ ID NO: 31 - DNA, 21 bp, Homo sapiens
  source 1..21: mol_type = genomic DNA
tggagaacagcctgttctcca

SEQ ID NO: 32 - DNA, 36 bp, Homo sapiens
  source 1..36: mol_type = genomic DNA
cacgtgtaatcccgtgggaggtcagacgtgcgtgtg

SEQ ID NO: 33 - DNA, 767 bp, Homo sapiens
  source 1..767: mol_type = genomic DNA
gtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgccctt

SEQ ID NO: 34 - DNA, 36 bp, Homo sapiens
  source 1..36: mol_type = genomic DNA
ggaggtcagacgtgcgtgtgcacgtgtaatcccgtg

SEQ ID NO: 35 - RNA, 15 nt, Homo sapiens
  source 1..15: mol_type = mRNA
accggtgccgccacc

SEQ ID NO: 36 - protein, 388 aa, Homo sapiens
MILWLHSLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 37 - DNA, 1167 bp, Homo sapiens
  source 1..1167: mol_type = genomic DNA
atgatactctggcttcacagtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 38 - protein, 644 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGEKFRVGNCKHLKMTRP

SEQ ID NO: 39 - DNA, 1935 bp, Homo sapiens
  source 1..1935: mol_type = genomic DNA
atggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctga

SEQ ID NO: 40 - DNA, 4176 bp, synthetic construct
  source 1..4176: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaaggtaagtctggaggcttgctttgggctgtatgctggaatcttccacctacttccctttttggcctctgactgaagggagtattggaagattccaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcactctctctctctgcagggtatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 41 - DNA, 4360 bp, synthetic construct
  source 1..4360: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactggaggcttgctttgggctgtatgctggaatcttccacctacttccctttttggcctctgactgaagggagtattggaagattccaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcacttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 42 - DNA, 4435 bp, synthetic construct
  source 1..4435: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggactggaggcttgctttgggctgtatgctggaatcttccacctacttccctttttggcctctgactgaagggagtattggaagattccaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcacttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 43 - DNA, 4251 bp, synthetic construct
  source 1..4251: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaaggtaagtctggaggcttgctttgggctgtatgctggaatcttccacctacttccctttttggcctctgactgaagggagtattggaagattccaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcactctctctctctgcagggtatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 44 - DNA, 4251 bp, synthetic construct
  source 1..4251: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaaggtaagtctggaggcttgctttgggctgtatgctgaatacttgaagggtagattcattttggcctctgactgatgaatctactttcaatattcaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcactctctctctctgcagggtatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 45 - DNA, 4251 bp, synthetic construct; identical to SEQ ID NO: 44
  source 1..4251: mol_type = other DNA

SEQ ID NO: 46 - DNA, 2580 bp, synthetic construct
  source 1..2580: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatgatactctggcttcacagtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtgaaggtaagtctggaggcttgctttgggctgtatgctgaatacttgaagggtagattcattttggcctctgactgatgaatctactttcaatattcaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcactctctctctctgcagggtatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 47 - DNA, 3348 bp, synthetic construct
  source 1..3348: mol_type = other DNA
tggagaacagcctgttctccacacgtgtaatcccgtgggaggtcagacgtgcgtgtggtaaaagtatctatgttcagaaagggaaatagaagctgaattaagtaattttatacacacgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccaccatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctgaaggtaagtctggaggcttgctttgggctgtatgctgaatacttgaagggtagattcattttggcctctgactgatgaatctactttcaatattcaggacacaaggcccttatcagcactctggaggcttgctttgggctgtatgctgttataacaggcagaagacatcttttggcctctgactgagatgtctcttctgttataacaggacacaaggcccttatcagcactctggaggcttgctttgggctgtatgctgtttaaaggcatacaacaccatttttggcctctgactgaatggtgtgttgcctttaaacaggacacaaggcccttatcagcactctctctctctgcagggtatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcgg

SEQ ID NO: 48 - DNA, 875 bp, synthetic construct
  source 1..875: mol_type = other DNA
tggagaacagcctgttctccacacgtgtaatcccgtgggaggtcagacgtgcgtgtggtaaaagtatctatgttcagaaagggaaatagaagctgaattaagtaattttatacaaccgttatggggtgcccttctgtgagtaattcttaaattcaaaagcaaatagttgtgaccacaaagggcttagtacatctcagctgattttagctctcagactcaaactggatatgattggtattgacctgctctgtgcatgtgattttaagatggtctgactcttgtctgtcattctgttgggttatattaataacaggggacaaagggtgaaataatccagtaaagttttgagcagccacttgacaacgtattccaaataaatgagaggaggaaaaccctagctctaccaactggggccataaaacagagtcttctacactctctttaatgtcaatattaaccagtttgcaaatgttcagtcagttatgggcatggatggatgtcctgaatgggttcccgggataaagtgtcatcataggacagaaatcacagggagagtgcaccaaggaaaaattacagtactgctatatttacttagtgcctctgaactagggttttattttccacgggttggaaagggaaccacctgtctcaattgctgatgtcagagagctccctcgagacacagggctgctggaaagcacatgatactgtacatatttgctcttacgttcgtatctggctaagattgggtttcagatttgtgccctattgtggagttcatttagtagtgactctgagatgccctcccacgtcaccatgcccttggaggtcagacgtgcgtgtgcacgtgtaatcccgtgaccggtgccgccacc

(19) United States
(12) Patent Application Publication
(10) Pub. No.: US 2026/0308598 A1
(43) Pub. Date: Oct. 8, 2026

(54) GENE THERAPY

(71) Applicant: CHI WAI WONG, Hong Kong (HK)

(72) Inventor: CHI WAI WONG, Hong Kong (HK)

(21) Appl. No.: 19/640,529

(22) Filed: Apr. 7, 2026

Related U.S. Application Data
(60) Provisional application No. 63/784,947, filed on Apr. 7, 2025.

(51) Int. Cl. C12N 15/113; A61K 38/17; A61K 48/00; A61P 25/28; C07K 14/72

(52) U.S. Cl. CPC C12N 15/1138; A61K 38/1796; A61K 48/005; A61P 25/28; C07K 14/721; C12N 2310/14

(57) ABSTRACT

The present disclosure provides gene therapies for diseases and conditions including but not limited to Kennedy's disease. Such gene therapies include the design of nucleic acid molecules, siRNA, compositions, plasmids, viruses, as well as related methods and uses.

### CROSS-REFERENCE TO RELATED APPLICATIONS

This application claims priority to the U.S. Provisional Application No. 63/784,947, filed on Apr. 7, 2025, the contents of which are hereby incorporated by reference.

### SEQUENCE LISTING

The instant application contains a Sequence Listing which has been submitted electronically in XML format and is hereby incorporated by reference in its entirety. The XML copy, created Apr. 1, 2026, is named “2026 Apr. 1-Sequence Listing-20938-0001US00” and is 87,644 bytes in size.

### TECHNICAL FIELD

The present disclosure generally relates to gene therapy, and in particular, to constructing nucleic acid molecules, double-stranded ribonucleic acid (dsRNA) agents, compositions, plasmids, or virus for the treatment of subjects suffering from Kennedy's disease.

### BACKGROUND

Kennedy's disease, also known as spinal-bulbar muscular atrophy (SBMA), is a rare neurodegenerative disorder caused by a mutation in the androgen receptor (AR) gene, which results in the expansion of cytosine-adenine-adenine (CAG) repeats within the AR gene. This mutation leads to an abnormal AR protein that accumulates in motor neurons, triggering cellular dysfunction, progressive muscle weakness, and atrophy. The disease predominantly affects males and is characterized by symptoms such as difficulty swallowing, speech impairments, and progressive loss of muscle function. Despite its severe impact on patients' quality of life, there are currently no approved therapies that specifically target the underlying molecular cause of Kennedy's disease.

Gene delivery techniques, such as viral vectors, are being explored for their potential to deliver molecules to target organs efficiently. This may allow for effective modulation of the disease-causing gene in affected individuals.

Accordingly, there is a pressing need for the development of therapeutic agents offering potential treatment options for Kennedy's disease. This disclosure addresses such needs by introducing novel nucleic acid-based therapies, specifically dsRNA agents, compositions, plasmids, and/or viruses designed to achieve these goals, providing a promising avenue for treating or preventing the progression of Kennedy's disease.

### SUMMARY

Embodiments of the present disclosure provide a nucleic acid molecule comprising a promoter module, an expression module, and a regulatory module wherein: the promoter module is configured to control transcription of the expression module and/or the regulatory module, the expression module includes a first gene of interest (GOI), which encodes a first target protein that is an exogenous protein to a cell, and the regulatory module includes one or more nucleotide segments that are configured to regulate a second GOI and/or a second target protein encoded by the second GOI, wherein the second target protein is an endogenous protein of the cell.

In some embodiments, the first target protein at least partially and functionally substitutes the second target protein in the cell.

In some embodiments, the first target protein at least partially and functionally attenuates the second target protein in the cell.

In some embodiments, the first GOI and the second GOI are not identical but have a sequence identity of at least 80%, 85%, 90%, 95%, or 98%.

In some embodiments, the first target protein is an androgen receptor.

In some embodiments, the first target protein is an androgen receptor (AR) that includes a polyglutamine (poly-Q) segment with less than 32 glutamines.

In some embodiments, the first target protein has one of amino acid sequences of SEQ ID NOs: 1, 36, or 38.

In some embodiments, the first GOI is an AR gene or its variants.

In some embodiments, the first GOI has one of DNA sequences of SEQ ID NO: 2, 37, or 39.

In some embodiments, the second target protein is an AR that causes spinal-bulbar muscular atrophy (SBMA).

In some embodiments, the second target protein is an AR that includes a polyglutamine (poly-Q) segment with at least 32 glutamines.

In some embodiments, the second target protein has an amino acid sequence of SEQ ID NO: 3.

In some embodiments, the second GOI has a DNA sequence of SEQ ID NO: 4.

In some embodiments, the regulatory module is configured to down-regulate an mRNA encoded by the second GOI.

In some embodiments, the one or more nucleotide segments are configured to target 5′-UTR or 3′-UTR of an mRNA encoded by the second GOI.

In some embodiments, the each of one or more nucleotide segments is configured to encode one or more a double stranded ribonucleic acid (dsRNA) agent.

In some embodiments, each of the one or more double stranded ribonucleic acid (dsRNA) agents includes a sense strand and an anti-sense strand.

In some embodiments, one of the one or more nucleotide segments encode an RNA sequence of SEQ ID NO: 5.

In some embodiments, one of the one or more nucleotide segments encode an RNA sequence of SEQ ID NO: 6.

In some embodiments, one of the one or more nucleotide segments encode an RNA sequence of SEQ ID NO: 7.

In some embodiments, one of the one or more nucleotide segments encode an RNA sequence of SEQ ID NO: 8.

In some embodiments, the promoter module includes a promoter that controls transcription of the expression module and the regulatory module.

In some embodiments, the promoter module responds to a physiological signal.

In some embodiments, the promoter module responds to an inflammatory signal.

In some embodiments, the promoter module includes a physiological signal responsive promoter sequence.

In some embodiments, the promoter module includes an Androgen Response Element (ARE) sequence.

In some embodiments, the promoter module includes an inflammatory response element.

In some embodiments, the promoter module includes a Kozak sequence.

In some embodiments, the nucleic acid molecule is a DNA molecule, and in a direction from 5′ to C 3′, comprises a nucleotide sequence as set forth in formula (I):

PromoterSeq-ExpressionSeq-RegulatorySeq (I), wherein PromoterSeq represents a nucleotide sequence of the promoter module; ExpressionSeq represents a nucleotide sequence of the expression module; and RegulatorySeq represents a nucleotide sequence of the regulatory module.

In some embodiments, the PromoterSeq has a nucleotide sequence that has at least 80%, 85%, 90%, 95%, 98%, or 99% identity to SEQ ID NO: 48.

In some embodiments, the PromoterSeq has a nucleotide sequence of SEQ ID NO: 48.

In some embodiments, The nucleic acid molecule has a nucleotide sequence that has at least 80%, 85%, 90%, 95%, 98%, or 99% identity to any one of SEQ ID NOs: 40-47.

In some embodiments, The nucleic acid molecule has a nucleotide sequence of any one of SEQ ID NOs: 40-47.

Embodiments of the present disclosure provide a double stranded ribonucleic acid (dsRNA) agent for inhibiting expression of androgen receptor (AR), comprising a sense strand and an antisense strand, wherein the sense strand comprises at least 21 contiguous nucleotides differing by no more than 5 nucleotides from any one of SEQ ID NOs: 13, 15, 17, 19, 21, 23, 25, 27, and 29, and the antisense strand comprises a nucleotide sequence that is at least partially complementary to the sense strand.

In some embodiments, the sense strand has a nucleotide sequence of SEQ ID NO: 13, 15, 17, 19, 21, 23, 25, 27, or 29 and the anti-sense RNA strand has a nucleotide sequence of SEQ ID NO: 14, 16, 18, 20, 22, 24, 26, 28, or 30.

In some embodiments, one or more nucleotides in the sense strand or the anti-sense strand are modified.

Embodiments of the present disclosure provide a pharmaceutical composition comprising the nucleic acid molecule or the dsRNA agent.

In some embodiments, the pharmaceutical composition further comprises a pharmaceutically acceptable carrier.

Embodiments of the present disclosure provide a plasmid comprising the nucleic acid molecule or a nucleotide sequence corresponding to the dsRNA agent. Embodiments of the present disclosure provide a virus comprising the nucleic acid molecule or a nucleotide sequence corresponding to the dsRNA agent.

In some embodiments, the virus is an adeno-associated virus (AAV).

Embodiments of the present disclosure provide a method for treating a subject, comprising administering the nucleic acid molecule, the dsRNA agent, the pharmaceutical composition, the plasmid, or the virus to the subject, wherein the subject is suffering from a disease.

In some embodiments, the disease is SBMA.

Embodiments of the present disclosure provide a use of the nucleic acid molecule, the dsRNA agent, the pharmaceutical composition, the plasmid, or the virus in the preparation of a medicament for use in the treatment of a subject suffering from a disease.

In some embodiments, the disease is SBMA.

### BRIEF DESCRIPTION OF THE DRAWINGS

The present disclosure is further described in terms of exemplary embodiments. These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1A is a schematic diagram illustrating the relative target gene (i.e., the second GOI) expressions of prostate cancer cells (22Rv1) transfected with si-NM0001 to si-NM0009, as well as a non-specific control siRNA (siR NC #1) according to the present disclosure;

FIG. 1B is a schematic diagram illustrating western blot of lysates from the prostate cancer cells transfected with siRNAs according to the present disclosure;

FIG. 2 is a schematic diagram illustrating relative target gene (i.e., the first GOI) expressions of the prostate cancer cells transfected with expression plasmids according to the present disclosure;

FIG. 3A is a schematic diagram illustrating relative target gene expressions of non-human primates (NHPs) infected with AAVs according to the present disclosure;

FIG. 3B is a schematic diagram illustrating western blot of lysates from muscle tissue of NHPs infected with AAV-0001 and AAV-0004 according to the present disclosure;

FIG. 4A is a schematic diagram illustrating NPPB gene expressions of induced polypotent stem cells (iPSCs) according to the present disclosure;

FIG. 4B is a schematic diagram illustrating NPPB gene expressions of iPSCs derived from an SBMA patient (SBMA-iPSCs) infected with the AAV-0001 according to the present disclosure;

FIG. 5A is a schematic diagram illustrating GPNMB gene expressions of the iPSCs according to the present disclosure;

FIG. 5B is a schematic diagram illustrating GPNMB gene expressions of the SBMA-iPSCs infected with the AAV-0001 according to the present disclosure;

FIG. 6A is a schematic diagram illustrating PDE7B gene expressions of the iPSCs according to the present disclosure;

FIG. 6B is a schematic diagram illustrating PDE7B gene expressions of the SBMA-iPSCs infected with the AAV-0001 according to the present disclosure;

FIG. 7A is a schematic diagram illustrating SHH gene expressions of the iPSCs according to the present disclosure; and

FIG. 7B is a schematic diagram illustrating SHH gene expressions of the SBMA-iPSCs infected with the AAV-0001 according to the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

### Definitions

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed disclosure, because the scope of the disclosure is limited only by the claims. Unless otherwise defined, all technical and scientific terms used herein have the same meaning as commonly understood by one of ordinary skill in the art to which this disclosure belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

As used herein, the term “RNA” includes RNA that encodes an amino acid sequence (e.g., mRNA, etc.) as well as RNA that does not encode an amino acid sequence (e.g., siRNA, shRNA, etc.). In some embodiments, an RNA can be an RNA that encodes an RNA, i.e., an RNA that encodes an amino acid sequence. Such RNA molecules, also called mRNA (messenger RNA), are single-stranded RNA molecules. In some embodiments, an RNA can be a non-coding RNA, i.e., an RNA that does not encode an amino acid sequence or is not translated into a protein. Non-coding RNAs may include, but are not limited to, small interfering RNAs (siRNAs), short or small hairpin RNAs (shRNAs), micrornas (miRNAs), piwi-interacting RNAs (piRNAs), and long non-coding RNAs (incRNAs). In some embodiments, an siRNA can comprise a double-stranded RNA (dsRNA) region, a hairpin structure, a loop structure, or a combination thereof. In some embodiments, as used herein, an siRNA may comprise at least one shRNA, at least one dsRNA region, or at least one loop structure. In some embodiments, as used herein, siRNA may be processed from dsRNA or shRNA. RNA can be prepared by synthetic chemical and enzymatic methods known to those of ordinary skill in the art, or by using recombinant techniques, or can be isolated from natural sources, or by a combination thereof. The RNA may optionally comprise non-natural and naturally occurring nucleoside modifications known in the art, e.g., N 1-methylpseuduridine, also referred to herein as methylpseuduridine.

As used herein, the term “expression” of a nucleic acid, gene, DNA, or RNA refers to both transcription and/or translation of the respective nucleic acid, gene, DNA, or RNA. The terms “modulate,” “increase,” “up-regulate,” “decrease,” and “down-regulate” expression of nucleic acid, gene, or target mRNA refer to altering the level of the protein encoded by the nucleic acid, gene by affecting the transcription and/or translation processes. For example, the terms “decreased”, “reduce”, “reduced”, “reduction”, “decrease”, and “inhibit” are all used herein generally refer to a statistically significant decrease in the expression level of a target gene to a reference or control such that the resulting protein level is diminished or absent. Such decrease may be measured at the mRNA, protein, or functional level and is considered statistically significant when compared to a baseline or untreated sample. In some embodiments, an extent of the reduction of the second target protein (e.g., AR that causes spinal-bulbar muscular atrophy (SBMA)) is sufficient to produce a therapeutic benefit, including, but not limited to, slowing the progression of Kennedy's disease or alleviating the severity of its symptoms.

As used herein, the terms “protein” and “peptide” are used to designate a series of amino acid residues each connected to each other by peptide bonds between the alpha-amino and carboxyl groups of adjacent residues. The terms “protein” and “peptide” also refer to a polymer of amino acids, including modified amino acids (e.g., phosphorylated, glycated, glycosylated, etc.) and amino acid analogs. In some embodiments, the terms “protein” and “peptide” are used interchangeably to refer to an encoded gene product, as well as any fragments thereof. Accordingly, exemplary proteins or peptides include gene products, naturally occurring proteins, homologs, orthologs, paralogs, and their respective fragments, as well as equivalents, variants, and analogs of these molecules.

The term “fragment” or “fragment of a sequence” has the same meaning herein, and is, for example, a shorter portion of the full-length sequence of a nucleic acid molecule, such as DNA or RNA, or a protein. Thus, a fragment typically consists of the same sequence as the corresponding segment within the full-length sequence.

As used herein, the term “vector” or “expression vector” refers to a naturally occurring or synthetically produced construct, e.g., a plasmid, miniloop, phagemid, cosmid, artificial chromosome/minichromosome, phage, virus such as baculovirus, retrovirus, adenovirus, adeno-associated virus, herpes simplex virus, phage, for uptake, propagation, expression or delivery of a nucleic acid in a cell.

The term “subject” or “patient” includes mammals. Examples of mammals include, but are not limited to, any member of the class mammalia: humans, non-human primates such as chimpanzees and other apes and monkey species; farm animals, such as cattle, horses, sheep, goats, pigs; domestic animals such as rabbits, dogs, and cats; laboratory animals, including rodents, such as rats, mice, and guinea pigs, and the like. In one aspect, the mammal is a human. As used herein, the term “animal” includes both human and non-human animals.

The terms “pharmaceutical composition” and “pharmaceutical formulation” (or “formulation”) are used interchangeably to refer to a mixture or solution comprising a therapeutically effective amount of an active pharmaceutical ingredient and one or more pharmaceutically acceptable excipients to be administered to a subject, such as a human in need thereof.

The term “pharmaceutically acceptable” refers to the properties of materials that can be used to prepare pharmaceutical compositions, which are generally safe, non-toxic, neither biologically nor otherwise undesirable, and acceptable for veterinary as well as human pharmaceutical use. “pharmaceutically acceptable” can refer to a material, such as a carrier or diluent, that does not abrogate the biological activity or properties of the compound, and is relatively non-toxic, i.e., the material can be administered to an individual without causing undesirable biological effects or interacting in a deleterious manner with any of the components of the composition in which it is contained.

The terms “pharmaceutically acceptable excipient”, “pharmaceutically acceptable carrier” and “therapeutically inert excipient” are used interchangeably and refer to any pharmaceutically acceptable ingredient in a pharmaceutical composition that is not therapeutically active and is non-toxic to the subject to which it is administered, such as disintegrants, binders, fillers, solvents, buffers, tonicity agents, stabilizers, antioxidants, surfactants, carriers, diluents, excipients, preservatives or lubricants used in formulating pharmaceutical products.

The terms “Kozak sequence” may refer to a nucleic acid sequence motif that serves as a translation start site for a protein.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or greater than 1 standard deviation, according to practice in the art. Alternatively, “about” may represent a range of up to 20%, up to 10%, up to 5%, or up to 1% of a given value. For example, if it is stated, “the compounds affected at least about 30% inhibition of androgen receptor”, it may be indicated that androgen receptor levels are inhibited within 25% to 35%.

“Contiguous nucleotides” means nucleotides immediately adjacent to each other.

“Complementarity” means the capacity for pairing between nucleobases of a first nucleic acid and a second nucleic acid.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Symptom(s)” of Kennedy's Disease include, but are not limited to, any one or more of the following: bulbar signs, such as difficult breathing, swallowing, or talking; dysphagia; intention tremor; hand tremors; lower motor neuropathy; muscle weakness and/or atrophy; muscle denervation; numbness or loss of sensation; decreased deep tendon reflexes; muscular fasciculations (e.g. unintentional muscle twitching); muscle cramps; muscle spasms; hypertrophied calf muscles; gynecomastia (enlarged breasts); effeminate effect of androgen deficiency; impotence; erectile dysfunction; reduced fertility; testicular atrophy; muscle asymmetry; and/or elevated serum creatine kinase.

“Treating Kennedy's Disease” refers to performing actions that lead to amelioration of Kennedy's Disease or of the symptoms accompanied therewith. The combination of said actions is encompassed by the term “treatment.” Amelioration of Kennedy's Disease includes, but is not limited to, increasing muscle strength, improving muscle atrophy, and/or inhibiting muscle denervation in a subject having Kennedy's Disease.

### DETAILED DESCRIPTIONS

The present disclosure provides a nucleic acid molecule comprising a promoter module, an expression module, and a regulatory module. In some embodiments, the promoter module is configured to control transcription of the expression module and/or the regulatory module. In some embodiments, the expression module includes a first gene of interest (GOI), which encodes a first target protein that is an exogenous protein to a cell. In some embodiments, the regulatory module includes one or more nucleotide segments that are configured to regulate a second GOI and/or a second target protein encoded by the second GOI, wherein the second target protein is an endogenous protein of the cell.

In some embodiments, the promoter module includes a promoter that control transcription of the expression module and the regulatory module. In some embodiments, the promoter module may be a constitutive promoter, allowing continuous expression of the downstream genes. Alternatively, the promoter may be an inducible promoter, which enables transcription only in the presence of a specific inducer, such as a chemical agent or environmental change. The promoter may also be tissue-specific, allowing targeted expression within a particular cell type or tissue. In some embodiments, the promoter may be an alternative promoter of PGC-1a.

Further details regarding the expression module and the regulatory module can be found in the following description.

In some embodiments, the promoter module responds to a physiological signal.

A physiological signal may refer to any biochemical or biophysical cue that is naturally present within the organism or cell, which can trigger the activation or repression of the promoter module.

In some embodiments, the promoter module may respond to various physiological signals, such as hormonal signals, nutrient availability, temperature-sensitive signals, and pH or ionic changes.

In some embodiments, the promoter module responds to an inflammatory signal.

The inflammatory signal may be defined as any molecular or biochemical cue that initiates, modulates, or sustains the inflammatory response within a cell, tissue, or organism. The inflammatory signal typically arises in response to infection, injury, or other forms of cellular stress, and activates signaling pathways that result in the expression of genes involved in immune cell recruitment, cytokine production, and the resolution or persistence of inflammation.

Inflammatory signals may include but are not limited to cytokines (e.g., TNF-α, IL-1, IL-6), reactive oxygen species (ROS), or the activation of pattern recognition receptors (PRRs) like toll-like receptors (TLRs), activate key transcription factors, including NF-κB, AP-1, and STAT proteins. The transcription factors may bind to specific DNA sequences within the promoter module, driving the expression of target genes involved in immune response, tissue repair, and inflammation.

In some embodiments, the promoter module includes a physiological signal responsive promoter sequence.

The signal responsive promoter sequence is a DNA sequence that allows the regulation of gene expression in response to specific physiological signals within a biological system. In some embodiments, the physiological signal responsive promoter sequence is shown as SEQ ID NO: 33:

GTAAAAGTATCTATGTTCAGAAAGGGAAATAGAAGCTGAATTAAGT
AATTTTATACACACGTTATGGGGTGCCCTTCTGTGAGTAATTCTT
AAATTCAAAAGCAAATAGTTGTGACCACAAAGGGCTTAGTACATC
TCAGCTGATTTTAGCTCTCAGACTCAAACTGGATATGATTGGTAT
TGACCTGCTCTGTGCATGTGATTTTAAGATGGTCTGACTCTTGTC
TGTCATTCTGTTGGGTTATATTAATAACAGGGGACAAAGGGTGAA
ATAATCCAGTAAAGTTTTGAGCAGCCACTTGACAACGTATTCCAA
ATAAATGAGAGGAGGAAAACCCTAGCTCTACCAACTGGGGCCATA
AAACAGAGTCTTCTACACTCTCTTTAATGTCAATATTAACCAGTT
TGCAAATGTTCAGTCAGTTATGGGCATGGATGGATGTCCTGAATG
GGTTCCCGGGATAAAGTGTCATCATAGGACAGAAATCACAGGGAG
AGTGCACCAAGGAAAAATTACAGTACTGCTATATTTACTTAGTGC
CTCTGAACTAGGGTTTTATTTTCCACGGGTTGGAAAGGGAACCAC
CTGTCTCAATTGCTGATGTCAGAGAGCTCCCTCGAGACACAGGGC
TGCTGGAAAGCACATGATACTGTACATATTTGCTCTTACGTTCGT
ATCTGGCTAAGATTGGGTTTCAGATTTGTGCCCTATTGTGGAGTT
CATTTAGTAGTGACTCTGAGATGCCCTCCCACGTCACCATGCCCT
T.

In some embodiments, the signal-responsive promoter sequence may include an Androgen Response Element (ARE) sequence.

The ARE sequence is a DNA sequence that can be recognized and bound by androgen receptors (ARs) in the presence of androgens, such as testosterone or dihydrotestosterone (DHT). The inclusion of an ARE sequence in the promoter module allows for the regulation of gene expression in response to androgen signaling, enabling precise control over gene activation or repression in tissues or cells that are responsive to androgens.

In some embodiments, the ARE sequence is shown as SEQ ID NO: 31:

TGGAGAACAGCCTGTTCTCCA.

In some embodiments, the promoter module may include an inflammatory response element (IRE).

In some embodiments, the IRE sequence is shown as SEQ ID NO: 32:

CACGTGTAATCCCGTGGGAGGTCAGACGTGCGTGTG.

In some embodiments, the IRE sequence is shown as SEQ ID NO: 34:

GGAGGTCAGACGTGCGTGTGCACGTGTAATCCCGTG.

In some embodiments, the promoter module may include a Kozak sequence.

In some embodiments, the Kozak sequence is shown as SEQ ID NO: 35:

ACCGGTGCCGCCACC.

The sequences listed above are exemplary only. In addition to the sequences provided, the promoter module may also include other element or sequence.

The expression module is a genetic construct designed to drive the expression of a gene of interest (GOI). In some embodiments, the expression module typically includes a coding sequence that contains the information to produce the target protein. In some embodiments, the expression module includes at least a first GOI, which encodes a first target protein that is an exogenous protein to the cell.

The regulatory module is a component of a genetic construct that functions to regulate the expression of one or more genes, typically the gene(s) of interest (GOI).

In some embodiments, the regulatory module of the nucleic acid molecule includes one or more nucleotide segments designed to regulate the expression of a second GOI. In some embodiments, the second GOI encodes a second target protein, which is an endogenous protein of the cell. In some embodiments, the regulatory module may includes regulatory elements such as enhancers, silencers, or other transcriptional regulators that modulate the expression of the second GOI. In some embodiments, the regulatory module is designed to downregulate or inhibit the expression of the second target protein.

In some embodiments, the first target protein is an androgen receptor.

The androgen receptor (AR) plays a critical role in the pathogenesis of spinal-bulbar muscular atrophy (SBMA), also known as Kennedy's disease, a rare neurodegenerative disorder. SBMA is caused by a mutation in the androgen receptor gene, specifically an expansion of CAG repeats in the coding region of the gene. This mutation leads to the production of a polyglutamine (polyQ) tract within the androgen receptor protein, which significantly affects the function and stability of the receptor, thereby contributing to the disease's characteristic symptoms.

In some embodiments, the first target protein is an androgen receptor (AR) that includes a polyglutamine (poly-Q) segment with less than 32 glutamines.

The poly-Q segment is defined by the number of consecutive glutamine (Q) amino acids within the androgen receptor protein, which may vary in length. In some embodiments, the first target protein is an AR that includes a poly-Q segment with 10 to 31 glutamines. In some embodiments, the first target protein is an AR that includes a poly-Q segment with 10 to 20 glutamines. In some embodiments, the first target protein is an AR that includes a poly-Q segment with 20 to 31 glutamines. In some embodiments, the first target protein is an AR that includes a poly-Q segment with 25 to 30 glutamines. In some embodiments, the first target protein is an AR that includes a poly-Q segment with 10 to 25 glutamines. In some embodiments, the first target protein may also encompass segments of any intermediate number of glutamine residues with 1 to 31 glutamines. In some embodiments, the first target protein is an AR that includes a poly-Q segment with 5, 10, 15, 20, 25, 28, 30, or 31 glutamines. In some embodiments, the first target protein is an AR that includes a poly-Q segment with 23 glutamines.

In some embodiments, the first target protein has one of amino acid sequences of SEQ ID NOs: 1, 36, or 38.

In some embodiments, the first GOI is an AR gene or its variants.

In some embodiments, the first GOI has one of DNA sequences of SEQ ID NO: 2, 37, or 39.

In some embodiments, the second target protein is an AR that causes spinal-bulbar muscular atrophy (SBMA).

In some embodiments, the second target protein is an AR that includes a polyglutamine (poly-Q) segment with at least 32 glutamines.

In some embodiments, the second target protein is an AR that includes a poly-Q segment with 32 to 40 glutamines. In some embodiments, the second target protein is an AR that includes a poly-Q segment with 40 to 50 glutamines. In some embodiments, the second target protein is an AR that includes a poly-Q segment with 50 to 60 glutamines. In some embodiments, the second target protein is an AR that includes a poly-Q segment with 60 to 70 glutamines. In some embodiments, the second target protein is an AR that includes a poly-Q segment with 32, 35, 40, 45, 50, 55, 60, 65, or 70 glutamines.

In some embodiments, the second target protein is an AR that includes a poly-Q segment with 48 glutamines.

In some embodiments, the second target protein has an amino acid sequence of SEQ ID NO: 3.

In some embodiments, the second GOI has a DNA sequence of SEQ ID NO: 4.

In some embodiments, the first target protein at least partially and functionally substitutes the second target protein in the cell.

As used herein, “partial substitution” may refer to the first target protein does not fully mimic the second target protein in every aspect, but can restore certain essential functions that are otherwise impaired or absent in the cell. For example, the first target protein may function in the same biochemical pathway or molecular process as the second target protein, but might not perform all of its roles with the same efficiency or under all conditions.

As used herein, “functional substitution” may refer to that the first target protein is capable of carrying out at least one critical biological activity of the second target protein. This can occur by the first protein mimicking the function of the second, either through structural similarity, shared binding partners, or the ability to perform similar enzymatic or regulatory functions.

In some embodiments, the first target protein performs a similar or complementary biological function to the second target protein, thereby compensating for its activity, function, or presence within the cellular environment.

For example, when the first target protein is androgen receptor, which could be a modified AR with a polyglutamine (poly-Q) segment of a certain length, may replace the activity of the second target protein, which may be an AR containing a longer poly-Q segment (e.g., associated with a disease condition such as spinal-bulbar muscular atrophy (SBMA)). The first target protein may bind to androgens and activate downstream gene expression similarly to the second target protein but without the pathological effects associated with polyglutamine expansion.

The term “at least partially and functionally substitutes” refers to that the first target protein does not need to fully replace all functions of the second target protein but can still perform key activities required by the cell, such as ligand binding, activation of target genes, or mediating specific cellular responses. For example, if the second target protein is a mutant androgen receptor (e.g., with a long poly-Q tract that causes aggregation and cellular toxicity), the first target protein could be a modified androgen receptor (with a shorter poly-Q tract) that retains the ability to respond to androgens and activate androgen-responsive genes, but without the detrimental aggregation and neurotoxicity.

In some embodiments, the first target protein at least partially and functionally attenuates the second target protein in the cell.

As used herein, “functional attenuation” refers to the reduction or suppression of the function of the second target protein by the first target protein.

For example, the first target protein AR may compete with the second target protein for binding to androgens or co-regulatory proteins, thus reducing the pathological activation of downstream signaling pathways that would normally occur due to the presence of the second target protein.

The term “at least partially and functionally attenuates” means that the first target protein does not necessarily eliminate the function of the second target protein completely, but it dampens or decreases its activity to a level that mitigates or alleviates undesirable cellular effects. For example, if the second target protein is an overactive or mutated protein contributing to a disease state, the first target protein may reduce its expression or activity without completely inhibiting it, thus allowing the cell to regain some level of normal function.

In some embodiments, the first GOI and the second GOI are not identical but have a sequence identity of at least 80%, 85%, 90%, 95%, or 98%.

In some embodiments, the regulatory module is configured to down-regulate the mRNA encoded by the second GOI. In some embodiments, the regulatory module includes one or more nucleotide segments that are configured to regulate the second GOI and/or the second target protein encoded by the second GOI, wherein the second target protein is the endogenous protein of the cell.

In some embodiments, the one or more nucleotide segments are configured to target 5′-UTR or 3′-UTR of an mRNA encoded by the second GOI.

In some embodiments, each of one or more nucleotide segments is configured to encode one or more a double stranded ribonucleic acid (dsRNA) agent.

In some embodiments, each of the one or more double stranded ribonucleic acid (dsRNA) agents includes a sense strand and an anti-sense strand.

In some embodiments, one of the one or more nucleotide segments encode an RNA sequence of SEQ ID NO: 5. In some embodiments, one of the one or more nucleotide segments encode an RNA sequence of SEQ ID NO: 6. In some embodiments, one of the one or more nucleotide segments encode an RNA sequence of SEQ ID NO: 7. In some embodiments, one of the one or more nucleotide segments encode an RNA sequence of SEQ ID NO: 8.

The regulatory module may include a variety of nucleotide sequences designed to modulate the expression and processing of the second gene of interest (GOI) and the second target protein. In some embodiments, the module may contain poly-A sequences (e.g., SEQ ID NO: 9, SEQ ID NO: 12), splice site donor and acceptor sequences (e.g., AGGTAAGT, SEQ ID NO: 11), and artificial miRNA sequences (e.g., SEQ ID NO: 5, SEQ ID NO: 6, SEQ ID NO: 7, SEQ ID NO: 8), which function at different levels of gene expression regulation, including mRNA stabilization, splicing, and post-transcriptional silencing.

Poly-A sequences are involved in enhancing mRNA stability and facilitating the addition of a poly-A tail to the mRNA, which is important for the regulation of transcription, mRNA export, and translation. The regulatory module may include one or more poly-A sequences.

Splice site sequences are essential for proper RNA splicing, which allows the removal of introns and the joining of exons to form mature mRNA. The regulatory module may include splice site donor and acceptor sequences that are important for correct mRNA processing.

Artificial miRNA sequences are designed to specifically target and regulate the expression of the second GOI or the second target protein. These miRNAs can induce RNA silencing or degradation, thereby providing a mechanism for post-transcriptional regulation. The regulatory module may include one or more artificial miRNA sequences to control the expression of the second GOI.

In some embodiments, the sequences contained in the regulatory module are as follows.

Poly-A Sequence (SEQ ID NO: 9):
CTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCA
AGGGGGAGGATTGGGAAGAGAATAGCAGGCATGCTGGGGA.
Poly-A Sequence (SEQ ID NO: 12):
TATTTGTGAAATTTGTGATGCTATTGCTTTATTTGTAACCATCTAGCTTTATTTGTGAAA
TTTGTGATGCTATTGCTTTATTTGTAACCATTATAAGCTGCAATAAACAAGTTAACAAC
AACAATTGCATTCATTTTATGTTTCAGGTTCAGGGGGAGATGTGGGAGGTTTTTTAA
AGCGG.
Splice site donor sequence:
AGGTAAGT.
Splice site acceptor sequence (SEQ ID NO: 11):
CTCTCTCTCTGCAGGG.
Artificial miRNA sequence: SEQ ID NO: 5:
CTGGAGGCTTGCTTTGGGCTGTATGCTGGAATCTTCCACCTACTTCCCTTTTTGGC
CTCTGACTGAAGGGAGTATTGGAAGATTCCAGGACACAAGGCCCTTATCAGCACT.
Artificial miRNA sequence (SEQ ID NO: 6):
CTGGAGGCTTGCTTTGGGCTGTATGCTGTTATAACAGGCAGAAGACATCTTTTGGC
CTCTGACTGAGATGTCTCTTCTGTTATAACAGGACACAAGGCCCTTATCAGCACT.
Artificial miRNA sequence (SEQ ID NO: 7):
CTGGAGGCTTGCTTTGGGCTGTATGCTGTTTAAAGGCATACAACACCATTTTTGGCC
TCTGACTGAATGGTGTGTTGCCTTTAAACAGGACACAAGGCCCTTATCAGCACT.
Artificial miRNA sequence (SEQ ID NO: 8):
CTGGAGGCTTGCTTTGGGCTGTATGCTGAATACTTGAAGGGTAGATTCATTTTGGCC
TCTGACTGATGAATCTACTTTCAATATTCAGGACACAAGGCCCTTATCAGCACT.

In some embodiments, the nucleic acid molecule is a DNA molecule, and in a direction from 5′ to C 3′, comprises a nucleotide sequence as set forth in formula (I):

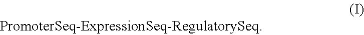

In some embodiments, for the nucleic acid molecule of formula (I), as well as the formulas shown below, PromoterSeq, ExpressionSeq, and RegulatorySeq each represents a segment sequence. In some embodiments, PromoterSeq represents a nucleotide sequence of the promoter module; ExpressionSeq represents a nucleotide sequence of the expression module; and RegulatorySeq represents a nucleotide sequence of the regulatory module.

In some embodiments, the PromoterSeq has a nucleotide sequence that has at least 80%, 85%, 90%, 95%, 98%, or 99% identity to SEQ ID NO: 48. In some embodiments, the PromoterSeq has a nucleotide sequence of SEQ ID NO: 48.

In some embodiments, the nucleic acid molecule has a nucleotide sequence that has at least 80%, 85%, 90%, 95%, 98%, or 99% identity to any one of SEQ ID NOs: 40-47. In some embodiments, the nucleic acid molecule has a nucleotide sequence of any one of SEQ ID NOs: 40-47.

The present disclosure provides a double stranded ribonucleic acid (dsRNA) agent for inhibiting expression of androgen receptor (AR). The dsRNA includes a sense strand and an antisense strand.

In some embodiments, the sense strand may be 13 to 33 nucleotides in length, and the antisense strand may be 16 to 33 nucleotides in length. The sense and antisense strands may be either the same length or different lengths. In some embodiments, the sense and antisense strands may be each independently 16 to 29 nucleotides in length. In some embodiments, both the sense and antisense strands may be each 18-26 nucleotides in length. In some embodiments, the sense and antisense strands may be each independently 21-23 nucleotides in length. In some embodiments, the sense strands may be each 13, 14, 15, 16, 17, 18, 19, 20, 21, 22, 23, 24, 25, 26, 27, 28, 29, 30, 31, 32, or 33 nucleotides in length. In some embodiments, the antisense strands may be each 16, 17, 18, 19, 20, 21, 22, 23, 24, 25, 26, 27, 28, 29, 30, 31, 32, or 33 nucleotides in length. In some embodiments, the sense strand may be about 19 nucleotides in length while the antisense may be about 21 nucleotides in length. In some embodiments, both the sense and antisense strands may be each 21 nucleotides in length. In some embodiments, the sense strand may be about 21 nucleotides in length while the antisense may be about 23 nucleotides in length.

The sense RNA strand may be identical to a portion of a mRNA sequence of a target gene (e.g., the AR gene). The anti-sense RNA strand may be complementary to the corresponding portion of the mRNA sequence of the target gene (e.g., the AR gene). In some embodiments, the sense RNA strand may be identical to a portion of a mRNA sequence of the second GOI. The anti-sense RNA strand may be complementary to the corresponding portion of the mRNA sequence of the second GOI. In some embodiments, the anti-sense RNA strand may be configured to identify the mRNA of the target gene and then bind thereto for initiating transcriptional silencing.

In some embodiments, the sense strand may comprise at least 21 contiguous nucleotides differing by no more than 5 nucleotides from any one of SEQ ID NOs: 13, 15, 17, 19, 21, 23, 25, 27, and 29, and the antisense strand may comprise a nucleotide sequence that is at least partially complementary to the sense strand. In some embodiments, the antisense RNA strand may have a nucleotide sequence having 70% or more (e.g., 75%, 80%, 85%, 87%, 90%, 92%, 95%, 97%, or 99%, or the like) complementary to the sense strand. In some embodiments, the antisense RNA strand may have a nucleotide sequence having at least 80% complementary to the sense strand. In some embodiments, the antisense RNA strand may have a nucleotide sequence having at least 90% complementary to the sense strand. In some embodiments, the antisense RNA strand may have a nucleotide sequence having at least 95% complementary to the sense strand. In some embodiments, the antisense RNA strand may have a nucleotide sequence that is fully complementary to the sense strand.

In some embodiments, the sense RNA strand may comprise at least 21 contiguous nucleotides differing by no more than 5 nucleotides from any one of SEQ ID NOs: 13, 15, 17, 19, 21, 23, 25, 27, and 29. It is noted that in some embodiments of the current disclosure, the terms “differ”, “differing”, or “different”, when applied to DNA/RNA sequences, does not include nucleotide modifications. For example, in the context of comparing sequences, when two RNA sequences have the same nucleotides as the same positions, these sequences are not considered to be different; two nucleotides, with or without chemical modifications noted below, that have the same nitrogenous base (e.g., A, C, T, G, or U), are not considered to be different. On the other hand, such limitation to these terms only applies in the context of sequence comparison.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 13. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 14. In some embodiments, the sense RNA strand may have a nucleotide sequence having at least 80% similarity with SEQ ID NO. 13, and the anti-sense RNA strand may have a nucleotide sequence having at least 80% similarity with SEQ ID NO. 14. In some embodiments, the sense RNA strand may have a nucleotide sequence having at least 90% similarity with SEQ ID NO. 13, and the anti-sense RNA strand may have a nucleotide sequence having at least 90% similarity with SEQ ID NO. 14. In some embodiments, the sense RNA strand may have a nucleotide sequence having at least 95% similarity with SEQ ID NO. 13, and the anti-sense RNA strand may have a nucleotide sequence having at least 95% similarity with SEQ ID NO. 14. In some embodiments, the sense strand may have a nucleotide sequence of SEQ ID NO: 13, and the anti-sense RNA strand may have a nucleotide sequence of SEQ ID NO: 14. The dsRNA agent includes SEQ ID NO. 13 and SEQ ID NO. 14 which may be referred to as si-NM0001 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 15. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 16. In some embodiments, the sense RNA strand may have a nucleotide sequence having at least 80% similarity with SEQ ID NO. 15, and the anti-sense RNA strand may have a nucleotide sequence having at least 80% similarity with SEQ ID NO. 16. In some embodiments, the sense RNA strand may have a nucleotide sequence having at least 90% similarity with SEQ ID NO. 15, and the anti-sense RNA strand may have a nucleotide sequence having at least 90% similarity with SEQ ID NO. 16. In some embodiments, the sense RNA strand may have a nucleotide sequence having at least 95% similarity with SEQ ID NO. 15, and the anti-sense RNA strand may have a nucleotide sequence having at least 95% similarity with SEQ ID NO. 16. In some embodiments, the sense strand may have a nucleotide sequence of SEQ ID NO: 15, and the anti-sense RNA strand may have a nucleotide sequence of SEQ ID NO: 16. The dsRNA agent includes SEQ ID NO. 15 and SEQ ID NO. 16 which may be referred to as si-NM0002 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 17. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 18. In some embodiments, the sense RNA strand may have a nucleotide sequence having at least 80% similarity with SEQ ID NO. 17, and the anti-sense RNA strand may have a nucleotide sequence having at least 80% similarity with SEQ ID NO. 18. In some embodiments, the sense RNA strand may have a nucleotide sequence having at least 90% similarity with SEQ ID NO. 17, and the anti-sense RNA strand may have a nucleotide sequence having at least 90% similarity with SEQ ID NO. 18. In some embodiments, the sense RNA strand may have a nucleotide sequence having at least 95% similarity with SEQ ID NO. 17, and the anti-sense RNA strand may have a nucleotide sequence having at least 95% similarity with SEQ ID NO. 18. In some embodiments, the sense strand may have a nucleotide sequence of SEQ ID NO: 17, and the anti-sense RNA strand may have a nucleotide sequence of SEQ ID NO: 18. The dsRNA agent includes SEQ ID NO. 17 and SEQ ID NO. 18 which may be referred to as si-NM0003 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 19. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 20. In some embodiments, the sense RNA strand may have a nucleotide sequence having at least 80% similarity with SEQ ID NO. 19, and the anti-sense RNA strand may have a nucleotide sequence having at least 80% similarity with SEQ ID NO. 20. In some embodiments, the sense RNA strand may have a nucleotide sequence having at least 90% similarity with SEQ ID NO. 19, and the anti-sense RNA strand may have a nucleotide sequence having at least 90% similarity with SEQ ID NO. 20. In some embodiments, the sense RNA strand may have a nucleotide sequence having at least 95% similarity with SEQ ID NO. 19, and the anti-sense RNA strand may have a nucleotide sequence having at least 95% similarity with SEQ ID NO. 20. In some embodiments, the sense strand may have a nucleotide sequence of SEQ ID NO: 19, and the anti-sense RNA strand may have a nucleotide sequence of SEQ ID NO: 20. The dsRNA agent includes SEQ ID NO. 19 and SEQ ID NO. 20 which may be referred to as si-NM0004 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 21. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 22. In some embodiments, the sense RNA strand may have a nucleotide sequence having at least 80% similarity with SEQ ID NO. 21, and the anti-sense RNA strand may have a nucleotide sequence having at least 80% similarity with SEQ ID NO. 22. In some embodiments, the sense RNA strand may have a nucleotide sequence having at least 90% similarity with SEQ ID NO. 21, and the anti-sense RNA strand may have a nucleotide sequence having at least 90% similarity with SEQ ID NO. 22. In some embodiments, the sense RNA strand may have a nucleotide sequence having at least 95% similarity with SEQ ID NO. 21, and the anti-sense RNA strand may have a nucleotide sequence having at least 95% similarity with SEQ ID NO. 22. In some embodiments, the sense strand may have a nucleotide sequence of SEQ ID NO: 21, and the anti-sense RNA strand may have a nucleotide sequence of SEQ ID NO: 22. The dsRNA agent includes SEQ ID NO. 21 and SEQ ID NO. 22 which may be referred to as si-NM0005 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 23. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 24. In some embodiments, the sense RNA strand may have a nucleotide sequence having at least 80% similarity with SEQ ID NO. 23, and the anti-sense RNA strand may have a nucleotide sequence having at least 80% similarity with SEQ ID NO. 24. In some embodiments, the sense RNA strand may have a nucleotide sequence having at least 90% similarity with SEQ ID NO. 23, and the anti-sense RNA strand may have a nucleotide sequence having at least 90% similarity with SEQ ID NO. 24. In some embodiments, the sense RNA strand may have a nucleotide sequence having at least 95% similarity with SEQ ID NO. 23, and the anti-sense RNA strand may have a nucleotide sequence having at least 95% similarity with SEQ ID NO. 24. In some embodiments, the sense strand may have a nucleotide sequence of SEQ ID NO: 23, and the anti-sense RNA strand may have a nucleotide sequence of SEQ ID NO: 24. The dsRNA agent includes SEQ ID NO. 23 and SEQ ID NO. 24 which may be referred to as si-NM0006 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 25. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 26. In some embodiments, the sense RNA strand may have a nucleotide sequence having at least 80% similarity with SEQ ID NO. 25, and the anti-sense RNA strand may have a nucleotide sequence having at least 80% similarity with SEQ ID NO. 26. In some embodiments, the sense RNA strand may have a nucleotide sequence having at least 90% similarity with SEQ ID NO. 25, and the anti-sense RNA strand may have a nucleotide sequence having at least 90% similarity with SEQ ID NO. 26. In some embodiments, the sense RNA strand may have a nucleotide sequence having at least 95% similarity with SEQ ID NO. 25, and the anti-sense RNA strand may have a nucleotide sequence having at least 95% similarity with SEQ ID NO. 26. In some embodiments, the sense strand may have a nucleotide sequence of SEQ ID NO: 25, and the anti-sense RNA strand may have a nucleotide sequence of SEQ ID NO: 26. The dsRNA agent includes SEQ ID NO. 25 and SEQ ID NO. 26 which may be referred to as si-NM0007 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 27. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 28. In some embodiments, the sense RNA strand may have a nucleotide sequence having at least 80% similarity with SEQ ID NO. 27, and the anti-sense RNA strand may have a nucleotide sequence having at least 80% similarity with SEQ ID NO. 28. In some embodiments, the sense RNA strand may have a nucleotide sequence having at least 90% similarity with SEQ ID NO. 27, and the anti-sense RNA strand may have a nucleotide sequence having at least 90% similarity with SEQ ID NO. 28. In some embodiments, the sense RNA strand may have a nucleotide sequence having at least 95% similarity with SEQ ID NO. 27, and the anti-sense RNA strand may have a nucleotide sequence having at least 95% similarity with SEQ ID NO. 28. In some embodiments, the sense strand may have a nucleotide sequence of SEQ ID NO: 27, and the anti-sense RNA strand may have a nucleotide sequence of SEQ ID NO: 28. The dsRNA agent includes SEQ ID NO. 27 and SEQ ID NO. 28 which may be referred to as si-NM0008 in Examples.

In some embodiments, the sense RNA strand may have a nucleotide sequence having 70% or more (e.g., 75%, 80%, 85%, 87%, 90%, 92%, 95%, 97%, or 99%, or the like) similarity with SEQ ID NO. 29. In some embodiments, the anti-sense RNA strand may have a nucleotide sequence having 70% or more (e.g., 75%, 80%, 85%, 87%, 90%, 92%, 95%, 97%, or 99%, or the like) similarity with SEQ ID NO. 30. In some embodiments, the sense RNA strand may have a nucleotide sequence having at least 80% similarity with SEQ ID NO. 29, and the anti-sense RNA strand may have a nucleotide sequence having at least 80% similarity with SEQ ID NO. 30. In some embodiments, the sense RNA strand may have a nucleotide sequence having at least 90% similarity with SEQ ID NO. 29, and the anti-sense RNA strand may have a nucleotide sequence having at least 90% similarity with SEQ ID NO. 30. In some embodiments, the sense RNA strand may have a nucleotide sequence having at least 95% similarity with SEQ ID NO. 29, and the anti-sense RNA strand may have a nucleotide sequence having at least 95% similarity with SEQ ID NO. 30. In some embodiments, the sense strand may have a nucleotide sequence of SEQ ID NO: 29, and the anti-sense RNA strand may have a nucleotide sequence of SEQ ID NO: 30. The dsRNA agent includes SEQ ID NO. 29 and SEQ ID NO. 30 which may be referred to as si-NM0009 in Examples.

In some embodiments, one or more nucleotides of the sense and/or anti-sense strands may be modified chemically to form modified nucleotide(s). Chemical modification of the nucleotide(s) may increase stability and/or specificity of the dsRNA, minimize immunogenicity of the dsRNA, and reduce off-target effect of the dsRNA.

In some embodiments, a modified nucleotide may include one or more of a deoxy-nucleotide, a 3′-terminal deoxythimidine (dT) nucleotide, a 2′-O-methyl modified nucleotide, a 2′-fluoro modified nucleotide, a phosphorothioate modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a thermally destabilizing nucleotide, a glycol modified nucleotide, and a 2-O—(N-methylacetamide) modified nucleotide. In some embodiments, a modified nucleotide may include one or more of a 2′-O-methyl modified nucleotide, a 2′-fluoro modified nucleotide, and a phosphorothioate modified nucleotide.

In some embodiments, different strategies of using these chemical modifications may be applied. For example, two or more chemical modifications may be used in combination (e.g., two or more chemical modifications may be performed on an individual nucleotide). As another example, one or more the chemical modifications may be used together with the phosphorothioate modification. As a further example, only one chemical modification may be used.

For brevity, the 2′-O-methyl modification may be represented be a letter of “m”, the phosphorothioate modification may be represented by a letter of “s”, and the 2′-fluoro modification may be represented be a letter of “f”. Merely by way of example, the modified nucleotides of the dsRNA may include a 2′-O-methyl modified guanine (i.e., mG), a 2′-O-methyl modified cytosine (i.e., mC), a 2′-O-methyl modified adenine (i.e., mA), and/or a 2′-O-methyl modified uracil (i.e., mU). As another example, the modified nucleotides of the dsRNA may include a phosphorothioate modified guanine (i.e., sG), a phosphorothioate modified cytosine (i.e., sC), a phosphorothioate modified adenine (i.e., sA), and/or a phosphorothioate modified uracil (i.e., sU). As still another example, the modified nucleotides of the dsRNA may include a 2′-fluoro modified guanine (i.e., fG), a 2′-fluoro modified cytosine (i.e., fC), a 2′-fluoro modified adenine (i.e., fA), and/or a 2′-fluoro modified uracil (i.e., fU). As a further example, the modified nucleotides of the dsRNA may include a 2′-O-methyl and phosphorothioate modified guanine (i.e., msG), a 2′-O-methyl and phosphorothioate modified cytosine (i.e., msC), a 2′-O-methyl and phosphorothioate modified adenine (i.e., msA), and/or a 2′-O-methyl and phosphorothioate modified uracil (i.e., msU). As still a further example, the modified nucleotides of the dsRNA may include a 2′-fluoro and phosphorothioate modified guanine (i.e., fsG), a 2′-fluoro and phosphorothioate modified cytosine (i.e., fsC), a 2′-fluoro and phosphorothioate modified adenine (i.e., fsA), and/or a 2′-fluoro and phosphorothioate modified uracil (i.e., fsU).

In some embodiments, part or all of the nucleotides of the sense RNA strand and/or the anti-sense RNA strand may be modified. In some embodiments, the sense RNA strand and/or the anti-sense RNA strand may include modified nucleotides at about 25%-100% of nucleotide positions. In some embodiments, the sense RNA strand and the anti-sense RNA strand may include modified nucleotides at about 60%-100% of nucleotide positions. In some embodiments, the sense RNA strand and the anti-sense RNA strand may include modified nucleotides at about 80%-100% of nucleotide positions. The sense RNA strand and the anti-sense RNA strand may include modified nucleotides at about 90%-100% of nucleotide positions. In some embodiments, the sense RNA strand and the anti-sense RNA strand may include modified nucleotides at 100% of nucleotide positions.

The present disclosure provides a pharmaceutical composition for inhibiting expression of androgen receptor (AR) comprising the nucleic acid molecule or the dsRNA agent. In some embodiments, a pharmaceutical composition further comprises a pharmaceutically acceptable carrier. The pharmaceutical composition is particularly useful in the inhibition of the expression of the AR mRNA in a target cell, a group of cells, a tissue, or an organism.

In some embodiments, the cell may be contacted with the nucleic acid molecule or the dsRNA agent in vitro or in vivo. In some embodiments, the cell may be within a subject.

In some embodiments, the cell may be a mammalian cell, e.g., a primate cell (such as a human cell, including human cell in a chimeric non-human animal, or a non-human primate cell, e.g., a monkey cell or a chimpanzee cell), or a non-primate cell. In some embodiments, the cell may be a human cell.

In some embodiments, the pharmaceutical composition may be used to treat or prevent a subject having a disease, disorder, or condition that would benefit from reduction in the level of the target AR mRNA, or inhibition in expression of the target gene. The pharmaceutical composition may be used to treat or prevent a subject at risk of developing a disease, disorder, symptom, or condition that would benefit from reduction of the level of the target mRNA or an inhibition in expression the target gene. In some embodiments, one or more pharmaceutically acceptable carriers (including vehicles, carriers, diluents, and/or delivery polymers) are added to the pharmaceutical composition that includes the dsRNA agent, thereby forming a pharmaceutical formulation or medicament suitable for in vivo delivery to a subject, including a human.

The pharmaceutical composition may include the dsRNA agent that can decrease the level of the AR mRNA in a cell, group of cells, group of cells, tissue, organ, or subject. In some embodiments, the subject has been previously identified as having a pathogenic upregulation of the target gene AR. In some embodiments, the subject has been previously identified or diagnosed as having Kennedy's disease. In some embodiments, the subject has been suffering from symptoms associated with Kennedy's disease. In some embodiments, the subject would benefit from a reduction of AR gene expression in the subject.

In some embodiments, the pharmaceutical composition including the dsRNA agent are used for treating or managing clinical presentations associated with the Kennedy's disease. In some embodiments, a therapeutically (including prophylactically) effective amount of one or more of pharmaceutical compositions is administered to a subject in need of such treatment. In some embodiments, administration of any of the dsRNA agent or the pharmaceutical composition may be used to decrease the number, severity, and/or frequency of symptoms of a disease in a subject.

The pharmaceutical composition including the nucleic acid molecule or the dsRNA agent may be used to treat at least one symptom in a subject having a disease or disorder that would benefit from reduction or inhibition in expression of AR mRNA.

In some embodiments, the subject is administered a therapeutically effective amount of one or more pharmaceutical compositions that include the nucleic acid molecule or the dsRNA agent thereby treating the symptom. In some embodiments, the subject is administered a prophylactically effective amount of one or more nucleic acid molecules or dsRNA agents, thereby preventing or inhibiting the at least one symptom.

The route of administration is the path by which the nucleic acid molecule or the dsRNA agent is brought into contact with the body. In general, methods of administering drugs and oligonucleotides and nucleic acids for treatment of a mammal arc well known in the art and may be applied to administration of the compositions. In some embodiments, the pharmaceutical composition may be administered via any suitable route in a preparation appropriately tailored to the particular route. The pharmaceutical composition may be administered by injection, for example, intravenously, intramuscularly, intracutaneously, subcutaneously, intraarticularly, or intraperitoneally. In some embodiments, the pharmaceutical composition may be administered via subcutaneous injection.

The pharmaceutical composition including the nucleic acid molecule or the dsRNA agent may be delivered to a cell, group of cells, tissue, or subject using oligonucleotide delivery. In general, any suitable method recognized in the art for delivering a nucleic acid molecule (in vitro or in vivo) may be adapted for use with the pharmaceutical composition. For example, delivery may be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the pharmaceutical composition may be administered by subcutaneous or intravenous infusion or injection.

In some embodiments, the pharmaceutical composition may be sterile. In some embodiment, the pharmaceutical composition may be pyrogen free.

In some embodiments, the pharmaceutical composition may include one or more pharmaceutically acceptable carriers. The pharmaceutical composition may be formulated for administration to the subject.

As used herein, the pharmaceutical composition or medicament includes a pharmacologically effective amount of at least one of the therapeutic compounds and one or more pharmaceutically acceptable carriers. Pharmaceutically acceptable carriers may be substances other than the Active Pharmaceutical Ingredient (API, therapeutic product) that are intentionally included in the drug delivery system. Carriers do not exert or are not intended to exert a therapeutic effect at the intended dosage. Carriers can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. The pharmaceutically acceptable carrier may or may not be an inert substance.

In some embodiments, the carrier may be absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

In some embodiments, the pharmaceutically acceptable carrier may be water for injection. In some embodiments, the pharmaceutically acceptable carrier may be isotonic saline.

The pharmaceutical composition suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Crcmophor® EL™ (BASF, Parsippany, NJ) or phosphate buffered saline (PBS). Suitable carriers should be stable under the conditions of manufacture and storage and should be preserved against the contaminating action of microorganisms such as bacteria and fungi. The carrier may be a solvent or dispersion medium containing, for example, water, ethanol, polyol (for example, glycerol, propylene glycol, and liquid polyethylene glycol), and suitable mixtures thereof. The proper fluidity may be maintained, for example, by the use of a coating such as lecithin, by the maintenance of the required particle size in the case of dispersion and by the use of surfactants. In some embodiments, it will be preferable to include isotonic agents, for example, sugars, polyalcohols such as mannitol, sorbitol, and sodium chloride in the composition. Prolonged absorption of the injectable composition may be brought about by including in the composition an agent which delays absorption, for example, aluminum monostearate and gelatin.

In some embodiments, the nucleic acid molecule or the dsRNA agent may be optionally combined with one or more additional therapeutics. The nucleic acid molecule or the dsRNA agent and additional therapeutic(s) may be administered in a single composition or they may be administered separately. In some embodiments, the one or more additional therapeutics are administered separately in separate dosage forms from the nucleic acid molecule or the dsRNA agent (e.g., the nucleic acid molecule or the dsRNA agent is administered by subcutaneous injection, while the additional therapeutic involved in the method of treatment dosing regimen is administered orally). In some embodiments, the nucleic acid molecule or the dsRNA agent may be administered to a subject in need thereof via subcutaneous injection, and the one or more optional additional therapeutics are administered orally, which together provide for a treatment regimen for diseases and conditions associated with Kennedy's disease. In some embodiments, the nucleic acid molecule or the dsRNA agent may be administered to the subject in need thereof via subcutaneous injection, and the one or more optional additional therapeutics may be administered via a separate subcutaneous injection. In some embodiments, the nucleic acid molecule or the dsRNA agent and one or more additional therapeutics may be combined into a single dosage form. The nucleic acid molecule or the dsRNA agents with or without the one or more additional therapeutics, may be combined with one or more carriers to form pharmaceutical compositions.

In some embodiments, an effective amount of the nucleic acid molecule or the dsRNA agent may be in the range of from about 0.1 to about 100 mg/kg of body weight per dose, e.g., from about 1.0 to about 50 mg/kg of body weight/dose. In some embodiments, an effective amount of the nucleic acid molecule or the dsRNA agent may be in the range of from about 0.25 to about 5 mg/kg of body weight per dose. In some embodiments, an effective amount of the nucleic acid molecule or the dsRNA agent may be in the range of from about 0.5 to about 6 mg/kg of body weight per dose. In some embodiments, an effective amount of the nucleic acid molecule or the dsRNA agent may be a fixed dose. In some embodiments, the fixed dose is in the range of from about 0.1 mg to about 1,000 mg of the nucleic acid molecule or the dsRNA agent. In some embodiments, the fixed does is in the range of 0.5 to 400 mg of the nucleic acid molecule or the dsRNA agent. In some embodiments, dosing may be weekly, bi-weekly, monthly, quarterly, or at any other interval depending on the dose of the nucleic acid molecule or the dsRNA agent administered, the activity level of the particular nucleic acid molecule or dsRNA agent, and the desired level of inhibition for the particular subject. The amount administered may depend on such variables as the overall health status of the patient or subject, the relative biological efficacy of the compound delivered, the formulation of the drug, the presence and types of excipients in the formulation, and the route of administration. The initial dosage administered may be increased beyond the above upper level to rapidly achieve the desired blood-level or tissue level, or the initial dosage may be smaller than the optimum.

The present disclosure provides a plasmid comprising the nucleic acid molecule or a nucleotide sequence corresponding to the dsRNA agent.

The present disclosure provides a virus comprising the nucleic acid molecule or a nucleotide sequence corresponding to the dsRNA agent. In some embodiments, the virus is an adeno-associated virus (AAV).

The present disclosure provides a method of treating a subject having a disorder or preventing the subject from suffering from the disorder that would benefit from reduction in AR expression, the method comprising administering to the subject a therapeutically effective amount of any one of the nucleic acid molecule, the dsRNA agent, the pharmaceutical composition, the plasmid, or the virus.

In some embodiments, the disorder may be an AR-associated disease. In some embodiments, the disease is SBMA.

The methods of the present disclosure may include administering to the subject a therapeutically effective amount of any one of the nucleic acid molecule, the dsRNA agent, the pharmaceutical composition, the plasmid, or the virus. The nucleic acid molecule, the dsRNA agent, the pharmaceutical composition, the plasmid, or the virus may be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In some embodiments, the nucleic acid molecule, the dsRNA agent, the pharmaceutical composition, the plasmid, or the virus may be administered by intravenous infusion or injection. In some embodiments, the nucleic acid molecule, the dsRNA agent, the pharmaceutical composition, the plasmid, or the virus may be administered by subcutaneous injection. In some embodiments, the nucleic acid molecule, the dsRNA agent, the pharmaceutical composition, the plasmid, or the virus may be administered by intramuscular injection.

In some embodiments, the administration may be via a depot injection. The depot injection may release the nucleic acid molecule, the dsRNA agent, the pharmaceutical composition, the plasmid, or the virus in a consistent way over a prolonged time period. Thus, the depot injection may reduce the frequency of dosing needed to obtain a desired effect, e.g., a desired inhibition of AR, or a therapeutic or prophylactic effect. The depot injection may also provide more consistent serum concentrations. The depot injection may include subcutaneous injections or intramuscular injections. In some embodiments, the depot injection may be a subcutaneous injection.

In some embodiments, the administration may be via a pump. The pump may be an external pump or a surgically implanted pump. In some embodiments, the pump may be a subcutaneously implanted osmotic pump. In some embodiments, the pump may be an infusion pump. The infusion pump may be used for intravenous, subcutaneous, arterial, or epidural infusions. In some embodiments, the infusion pump may be a subcutaneous infusion pump.

The mode of administration may be chosen based on whether local or systemic treatment is desired and based on the area to be treated. The route and site of administration may be chosen to enhance targeting.

In some embodiments, the method includes administering the nucleic acid molecule, the dsRNA agent, the pharmaceutical composition, the plasmid, or the virus featured herein such that expression of the AR gene is decreased, for example, over a period of about 0.5, 1, 2, 3, 4, 5, 6, 1-6, 1-3, or 3-6 months per dose. In some embodiments, the nucleic acid molecule, the dsRNA agent, the pharmaceutical composition, the plasmid, or the virus is administered once every 1-6 months.

In some embodiments, the subject may be administered a therapeutic amount of the nucleic acid molecule, the dsRNA agent, the pharmaceutical composition, the plasmid, or the virus about 0.01 mg/kg to about 200 mg/kg. In some embodiments, the subject may be administered a therapeutic amount of the nucleic acid molecule, the dsRNA agent, the pharmaceutical composition, the plasmid, or the virus about 3 mg to about 1000 mg as a fixed dose, regardless of body weight.

In some embodiments, the nucleic acid molecule, the dsRNA agent, the pharmaceutical composition, the plasmid, or the virus is administered by subcutaneous injection. One or more injections may be used to deliver the desired dose of the nucleic acid molecule or dsRNA to a subject. The injections may be repeated over a period of time.

The administration may be repeated on a regular basis. In some embodiments, after an initial treatment regimen, the treatments may be administered on a less frequent basis. A repeat-dose regimen may include administration of a therapeutic amount of dsRNA on a regular basis, such as once per month to once a year. In some embodiments, the nucleic acid molecule, the dsRNA agent, the pharmaceutical composition, the plasmid, or the virus is administered about once per month to about once every three months, or about once every three months to about once every six months.

The present disclosure provides a use of the chimeric protein of any one of the nucleic acid molecule, the dsRNA agent, the pharmaceutical composition, the plasmid, or the virus in the preparation of a medicament for use in the treatment of a subject suffering from a disease, wherein the disease can be treated or prevented by a reduction in AR expression.

The use of the chimeric protein of any one of the nucleic acid molecule, the dsRNA agent, the pharmaceutical composition, the plasmid, or the virus of the disclosure enables the targeted degradation of mRNAs of the corresponding gene (AR gene) in a subject. Using in vitro and in vivo assays, examples have demonstrated that the chimeric protein targeting the AR gene can potently mediate RNA, resulting in significant inhibition of expression of the AR gene. Thus, methods and compositions including these chimeric protein are useful for treating a subject having the AR-associated disease. In some embodiments, the disease is SBMA.

In some embodiments of the current invention, the simultaneous inhibition of an endogenous protein (e.g., an abnormal version of a functional protein) and introduction of an exogenous version of that protein (e.g., the normal version) can be achieved. Taking Kinnedy's disease as an example, the AR protein in a patient's cells includes prolonged poly-Q sequences and causes various symptoms; with the current invention, the expression of such high-poly-Q endogenous proteins can be inhibited and a low-poly-Q exogenous version of the AR can be introduced to the functioning cells, which may result in a reduction of the symptoms and improve of the patient's quality of life. In addition, the current invention is not limited to the application in Kinnedy's disease. Any schemes that fit the basic design of the present disclosure would fall within the bounds of the current invention.

This disclosure is further illustrated by the following examples which should not be construed as limiting.

### EXAMPLES

### Example 1: Design siRNAs

SiRNAs targeting 5′UTR or 3′UTR of mRNAs encoding an endogenous gene of interest (i.e., the second GOI) were designed. Specifically, si-NM0001, si-NM0002, si-NM0003, si-NM0004, si-NM0005, si-NM0006, si-NM0007, si-NM0008, and si-NM0009 targeting either the 5′UTR or 3′UTR of androgen receptor mRNAs. The constructed sequences are listed in Table 1.

TABLE 1
Sequences of siRNAs and GOI

| Description | Sequence |
|---|---|
| si- | Sense strand 5′-AGGGAAGTAGGTGGAAGATTC-3′(SEQ ID NO: 13) |
| NM0001 | Anti-sense strand 5′-GAATCTTCCACCTACTTCCCT-3′(SEQ ID NO: 14) |
| si- | Sense strand 5′-GATGTCTTCTGCCTGTTATAA-3′(SEQ ID NO: 15) |
| NM0002 | Anti-sense strand 5′-TTATAACAGGCAGAAGACATC-3′(SEQ ID NO: 16) |
| si- | Sense strand 5′-ATGGTGTTGTATGCCTTTAAA-3′(SEQ ID NO: 17) |
| NM0003 | Anti-sense strand 5′-TTTAAAGGCATACAACACCAT-3′(SEQ ID NO: 18) |
| si- | Sense strand 5′-TGAATCTACCCTTCAAGTATT-3′(SEQ ID NO: 19) |
| NM0004 | Anti-sense strand 5′-AATACTTGAAGGGTAGATTCA-3′(SEQ ID NO: 20) |
| si- | Sense strand 5′-CATTGCAAAGAAGGCTCTTAG-3′(SEQ ID NO: 21) |
| NM0005 | Anti-sense strand 5′-CTAAGAGCCTTCTTTGCAATG-3′(SEQ ID NO: 22) |
| si- | Sense strand 5′-CTTCTTCTGCACGAGACTTTG-3′(SEQ ID NO: 23) |
| NM0006 | Anti-sense strand 5′-CAAAGTCTCGTGCAGAAGAAG-3′(SEQ ID NO: 24) |
| si- | Sense strand 5′-CCCTCCACGGGAGACTTTATT-3′(SEQ ID NO: 25) |
| NM0007 | Anti-sense strand 5′-AATAAAGTCTCCCGTGGAGGG-3′(SEQ ID NO: 26) |
| si- | Sense strand 5′-ACTGTGGAGCAATTCATTATA-3′(SEQ ID NO: 27) |
| NM0008 | Anti-sense strand 5′-TATAATGAATTGCTCCACAGT-3′(SEQ ID NO: 28) |
| si- | Sense strand 5′-AGAAGGAGAGCAGAGATTTAA-3′(SEQ ID NO: 29) |
| NM0009 | Anti-sense strand 5′-TTAAATCTCTGCTCTCCTTCT-3′(SEQ ID NO: 30) |
| endogenous | MEVQLGLGRVYPRPPSKTYRGAFQNLFQSVREVIQNPGPRHPEAASAAP |
| androgen | PGASLLLLQQQQQQQQQQQQQQQQQQQQQQQQQQQQQQQQQQQQQ |
| receptor | QQQQQQQQQQQETSPRQQQQQQGEDGSPQAHRRGPTGYLVLDEEQ |
| | QPSQPQSALECHPERGCVPEPGAAVAASKGLPQQLPAPPDEDDSA |
| | APSTLSLLGPTFPGLSSCSADLKDILSEASTMQLLQQQQQEAVSE |
| | GSSSGRAREASGAPTSSKDNYLGGTSTISDNAKELCKAVSVSMGL |
| | GVEALEHLSPGEQLRGDCMYAPLLGVPPAVRPTPCAPLAECKGSL |
| | LDDSAGKSTEDTAEYSPFKGGYTKGLEGESLGCSGSAAAGSSGTL |
| | ELPSTLSLYKSGALDEAAAYQSRDYYNFPLALAGPPPPPPPPHPH |
| | ARIKLENPLDYGSAWAAAAAQCRYGDLASLHGAGAAGPGSGSPSA |
| | AASSSWHTLFTAEEGQLYGPCGGGGGGGGGGGGGGGGGGGGGGGE |
| | AGAVAPYGYTRPPQGLAGQESDFTAPDVWYPGGMVSRVPYPSPTC |
| | VKSEMGPWMDSYSGPYGDMRLETARDHVLPIDYYFPPQKTCLICG |
| | DEASGCHYGALTCGSCKVFFKRAAEGKQKYLCASRNDCTIDKFRR |
| | KNCPSCRLRKCYEAGMTLGARKLKKLGNLKLQEEGEASSTTSPTE |
| | ETTQKLTVSHIEGYECQPIFLNVLEAIEPGVVCAGHDNNQPDSFA |
| | ALLSSLNELGERQLVHVVKWAKALPGFRNLHVDDQMAVIQYSWMG |
| | LMVFAMGWRSFTNVNSRMLYFAPDLVFNEYRMHKSRMYSQCVRMR |
| | HLSQEFGWLQITPQEFLCMKALLLFSIIPVDGLKNQKFFDELRMN |
| | YIKELDRIIACKRKNPTSCSRRFYQLTKLLDSVQPIARELHQFTF |
| | DLLIKSHMVSVDFPEMMAEIISVQVPKILSGKVKPIYFHTQ |
| | (SEQ ID NO: 3) |
| the | ATGGAAGTGCAGTTAGGGCTGGGAAGGGTCTACCCTCGGCCGCCG |
| TGA | TCCAAGACCTACCGAGGAGCTTTCCAGAATCTGTTCCAGAGCGTG |
| second | CGCGAAGTGATCCAGAACCCGGGCCCCAGGCACCCAGAGGCCGCG |
| GOI | AGCGCAGCACCTCCCGGCGCCAGTTTGCTGCTGCTGCAGCAGCAG |
| | CAGCAGCAGCAGCAGCAGCAGCAGCAGCAGCAGCAGCAGCAGCAG |
| | CAGCAGCAGCAGCAGCAGCAGCAGCAGCAGCAGCAGCAGCAGCAG |
| | CAGCAGCAGCAGCAGCAGCAGCAGCAGCAGCAGCAGCAGCAGCAA |
| | GAGACTAGCCCCAGGCAGCAGCAGCAGCAGCAGGGTGAGGATGGT |
| | TCTCCCCAAGCCCATCGTAGAGGCCCCACAGGCTACCTGGTCCTG |
| | GATGAGGAACAGCAACCTTCACAGCCGCAGTCGGCCCTGGAGTGC |
| | CACCCCGAGAGAGGTTGCGTCCCAGAGCCTGGAGCCGCCGTGGCC |
| | GCCAGCAAGGGGCTGCCGCAGCAGCTGCCAGCACCTCCGGACGAG |
| | GATGACTCAGCTGCCCCATCCACGTTGTCCCTGCTGGGCCCCACT |
| | TTCCCCGGCTTAAGCAGCTGCTCCGCTGACCTTAAAGACATCCTG |
| | AGCGAGGCCAGCACCATGCAACTCCTTCAGCAACAGCAGCAGGAA |
| | GCAGTATCCGAAGGCAGCAGCAGCGGGAGAGCGAGGGAGGCCTCG |
| | GGGGCTCCCACTTCCTCCAAGGACAATTACTTAGGGGGCACTTCG |
| | ACCATTTCTGACAACGCCAAGGAGTTGTGTAAGGCAGTGTCGGTG |
| | TCCATGGGCCTGGGTGTGGAGGCGTTGGAGCATCTGAGTCCAGGG |
| | GAACAGCTTCGGGGGGATTGCATGTACGCCCCACTTTTGGGAGTT |
| | CCACCCGCTGTGCGTCCCACTCCTTGTGCCCCATTGGCCGAATGC |
| | AAAGGTTCTCTGCTAGACGACAGCGCAGGCAAGAGCACTGAAGAT |
| | ACTGCTGAGTATTCCCCTTTCAAGGGAGGTTACACCAAAGGGCTA |
| | GAAGGCGAGAGCCTAGGCTGCTCTGGCAGCGCTGCAGCAGGGAGC |
| | TCCGGGACACTTGAACTGCCGTCTACCCTGTCTCTCTACAAGTCC |
| | GGAGCACTGGACGAGGCAGCTGCGTACCAGAGTCGCGACTACTAC |
| | AACTTTCCACTGGCTCTGGCCGGACCGCCGCCCCCTCCGCCGCCT |
| | CCCCATCCCCACGCTCGCATCAAGCTGGAGAACCCGCTGGACTAC |
| | GGCAGCGCCTGGGCGGCTGCGGCGGCGCAGTGCCGCTATGGGGAC |
| | CTGGCGAGCCTGCATGGCGCGGGTGCAGCGGGACCCGGTTCTGGG |
| | TCACCCTCAGCCGCCGCTTCCTCATCCTGGCACACTCTCTTCACA |
| | GCCGAAGAAGGCCAGTTGTATGGACCGTGTGGTGGTGGTGGGGGT |
| | GGTGGCGGCGGCGGCGGCGGCGGCGGCGGCGGCGGCGGCGGCGGC |
| | GGCGGCGGCGAGGCGGGAGCTGTAGCCCCCTACGGCTACACTCGG |
| | CCCCCTCAGGGGCTGGCGGGCCAGGAAAGCGACTTCACCGCACCT |
| | GATGTGTGGTACCCTGGCGGCATGGTGAGCAGAGTGCCCTATCCC |
| | AGTCCCACTTGTGTCAAAAGCGAAATGGGCCCCTGGATGGATAGC |
| | TACTCCGGACCTTACGGGGACATGCGTTTGGAGACTGCCAGGGAC |
| | CATGTTTTGCCCATTGACTATTACTTTCCACCCCAGAAGACCTGC |
| | CTGATCTGTGGAGATGAAGCTTCTGGGTGTCACTATGGAGCTCTC |
| | ACATGTGGAAGCTGCAAGGTCTTCTTCAAAAGAGCCGCTGAAGGG |
| | AAACAGAAGTACCTGTGCGCCAGCAGAAATGATTGCACTATTGAT |
| | AAATTCCGAAGGAAAAATTGTCCATCTTGTCGTCTTCGGAAATGT |
| | TATGAAGCAGGGATGACTCTGGGAGCCCGGAAGCTGAAGAAACTT |
| | GGTAATCTGAAACTACAGGAGGAAGGAGAGGCTTCCAGCACCACC |
| | AGCCCCACTGAGGAGACAACCCAGAAGCTGACAGTGTCACACATT |
| | GAAGGCTATGAATGTCAGCCCATCTTTCTGAATGTCCTGGAAGCC |
| | ATTGAGCCAGGTGTAGTGTGTGCTGGACACGACAACAACCAGCCC |
| | GACTCCTTTGCAGCCTTGCTCTCTAGCCTCAATGAACTGGGAGAG |
| | AGACAGCTTGTACACGTGGTCAAGTGGGCCAAGGCCTTGCCTGGC |
| | TTCCGCAACTTACACGTGGACGACCAGATGGCTGTCATTCAGTAC |
| | TCCTGGATGGGGCTCATGGTGTTTGCCATGGGCTGGCGATCCTTC |
| | ACCAATGTCAACTCCAGGATGCTCTACTTCGCCCCTGATCTGGTT |
| | TTCAATGAGTACCGCATGCACAAGTCCCGGATGTACAGCCAGTGT |
| | GTCCGAATGAGGCACCTCTCTCAAGAGTTTGGATGGCTCCAAATC |
| | ACCCCCCAGGAATTCCTGTGCATGAAAGCACTGCTACTCTTCAGC |
| | ATTATTCCAGTGGATGGGCTGAAAAATCAAAAATTCTTTGATGAA |
| | CTTCGAATGAACTACATCAAGGAACTCGATCGTATCATTGCATGC |
| | AAAAGAAAAAATCCCACATCCTGCTCAAGACGCTTCTACCAGCTC |
| | ACCAAGCTCCTGGACTCCGTGCAGCCTATTGCGAGAGAGCTGCAT |
| | CAGTTCACTTTTGACCTGCTAATCAAGTCACACATGGTGAGCGTG |
| | GACTTTCCGGAAATGATGGCAGAGATCATCTCTGTGCAAGTGCCC |
| | AAGATCCTTTCTGGGAAAGTCAAGCCCATCTATTTCCACACCCAG |
| | (SEQ ID NO: 4) |

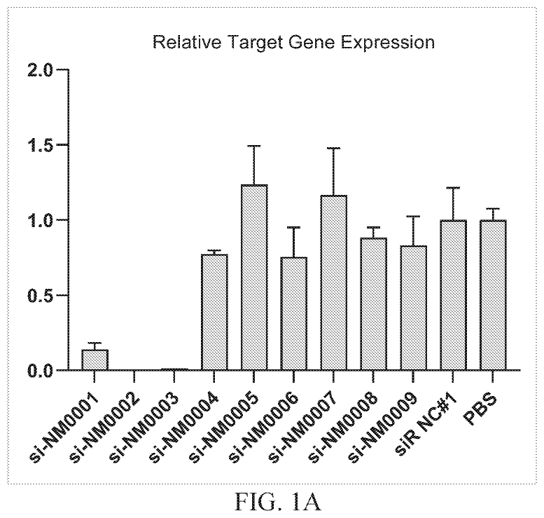

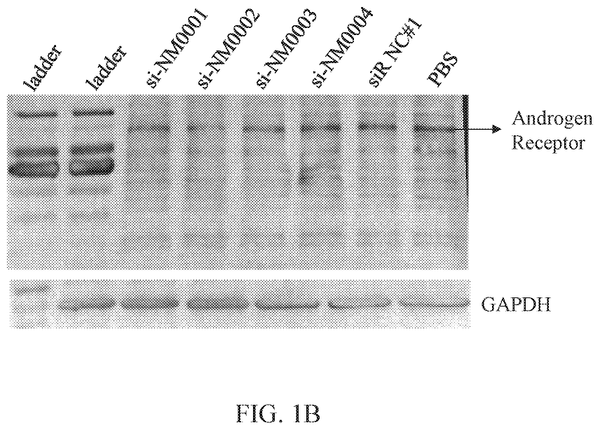

Cell lines were transfected with the siRNAs and expression levels of the second GOI were detected by qRT-PCR. FIG. 1A is a schematic diagram illustrating the relative target gene (i.e., the second GOI) expressions of prostate cancer cells (22Rv1) transfected with si-NM0001 to si-NM0009, as well as a non-specific control siRNA (siR NC #1) according to the present disclosure. As shown in FIG. 1A, the mRNA expression levels of the second GOI are significantly suppressed by the si-NM0001, the si-NM0002, and the si-NM0003.

Western blot of lysates from the prostate cancer cells transfected with the siRNAs were analyzed for the androgen receptor compared to PBS and the siR NC #1 controls. FIG. 1B is a schematic diagram illustrating the Western blot of lysates from the prostate cancer cells transfected with the siRNAs according to the present disclosure. As shown in FIG. 1B, the si-NM0001, the si-NM0002, and the si-NM0003 modestly reduced the protein expression level of the androgen receptor compared to the PBS or the siR NC #1 controls.

### Example 2: Design Expression Plasmids

Expression plasmids were designed and constructed using a promoter which can be regulated by physiological signals or small chemical molecules. In some embodiments, the promoter may be an alternative promoter of PGC-1a with an inflammation response element and an androgen response element added onto.

Expression cassettes encoding the exogenous gene of interest (i.e. the first GOI) and an artificial miRNA targeting endogenous androgen receptor mRNAs encoded by the second GOI or other genes of interest may be cloned into the expression plasmid. In some embodiments, the first GOI may be an androgen receptor devoid of its endogenous 5′UTR and 3′UTR; whereas, the artificial miRNA may be using guiding sequences based on the si-NM0001, the si-NM0002, and the si-NM0003 targeting the 5′UTR or 3′UTR of the endogenous androgen receptor mRNAs. The constructed sequences are listed in Table 2.

TABLE 2
Sequences of expression plasmids

| Description | Sequence |
|---|---|
| promoter | TGGAGAACAGCCTGTTCTCCACACGTGTAATCCCGTGGGAGGTCA |
| | GACGTGCGTGTGGTAAAAGTATCTATGTTCAGAAAGGGAAATAGAA |
| | GCTGAATTAAGTAATTTTATACACACGTTATGGGGTGCCCTTCTGTGA |
| | GTAATTCTTAAATTCAAAAGCAAATAGTTGTGACCACAAAGGGCTTA |
| | GTACATCTCAGCTGATTTTAGCTCTCAGACTCAAACTGGATATGATT |
| | GGTATTGACCTGCTCTGTGCATGTGATTTTAAGATGGTCTGACTCTT |
| | GTCTGTCATTCTGTTGGGTTATATTAATAACAGGGGACAAAGGGTGA |
| | AATAATCCAGTAAAGTTTTGAGCAGCCACTTGACAACGTATTCCAAA |
| | TAAATGAGAGGAGGAAAACCCTAGCTCTACCAACTGGGGCCATAAA |
| | ACAGAGTCTTCTACACTCTCTTTAATGTCAATATTAACCAGTTTGCAA |
| | ATGTTCAGTCAGTTATGGGCATGGATGGATGTCCTGAATGGGTTCC |
| | CGGGATAAAGTGTCATCATAGGACAGAAATCACAGGGAGAGTGCAC |
| | CAAGGAAAAATTACAGTACTGCTATATTTACTTAGTGCCTCTGAACTA |
| | GGGTTTTATTTTCCACGGGTTGGAAAGGGAACCACCTGTCTCAATT |
| | GCTGATGTCAGAGAGCTCCCTCGAGACACAGGGCTGCTGGAAAGC |
| | ACATGATACTGTACATATTTGCTCTTACGTTCGTATCTGGCTAAGATT |
| | GGGTTTCAGATTTGTGCCCTATTGTGGAGTTCATTTAGTAGTGACTC |
| | TGAGATGCCCTCCCACGTCACCATGCCCTTGGAGGTCAGACGTGC |
| | GTGTGCACGTGTAATCCCGTGACCGGTGCCGCCACC |
| | (SEQ ID NO: 48) |
| exogenous | MEVQLGLGRVYPRPPSKTYRGAFQNLFQSVREVIQNPGPRHPEAASA |
| androgen | APPGASLLLLQQQQQQQQQQQQQQQQQQQQQQQETSPRQQQQQ |
| receptor | QGEDGSPQAHRRGPTGYLVLDEEQQPSQPQSALECHPERGCVPEPG |
| | AAVAASKGLPQQLPAPPDEDDSAAPSTLSLLGPTFPGLSSCSADLKDIL |
| | SEASTMQLLQQQQQEAVSEGSSSGRAREASGAPTSSKDNYLGGTSTI |
| | SDNAKELCKAVSVSMGLGVEALEHLSPGEQLRGDCMYAPLLGVPPAV |
| | RPTPCAPLAECKGSLLDDSAGKSTEDTAEYSPFKGGYTKGLEGESLG |
| | CSGSAAAGSSGTLELPSTLSLYKSGALDEAAAYQSRDYYNFPLALAGP |
| | PPPPPPPHPHARIKLENPLDYGSAWAAAAAQCRYGDLASLHGAGAAG |
| | PGSGSPSAAASSSWHTLFTAEEGQLYGPCGGGGGGGGGGGGGGG |
| | GGGGGGGGEAGAVAPYGYTRPPQGLAGQESDFTAPDVWYPGGMVS |
| | RVPYPSPTCVKSEMGPWMDSYSGPYGDMRLETARDHVLPIDYYFPP |
| | QKTCLICGDEASGCHYGALTCGSCKVFFKRAAEGKQKYLCASRNDCTI |
| | DKFRRKNCPSCRLRKCYEAGMTLGARKLKKLGNLKLQEEGEASSTTS |
| | PTEETTQKLTVSHIEGYECQPIFLNVLEAIEPGVVCAGHDNNQPDSFAA |
| | LLSSLNELGERQLVHVVKWAKALPGFRNLHVDDQMAVIQYSWMGLMV |
| | FAMGWRSFTNVNSRMLYFAPDLVFNEYRMHKSRMYSQCVRMRHLSQ |
| | EFGWLQITPQEFLCMKALLLFSIIPVDGLKNQKFFDELRMNYIKELDRII |
| | ACKRKNPTSCSRRFYQLTKLLDSVQPIARELHQFTFDLLIKSHMVSVDF |
| | PEMMAEIISVQVPKILSGKVKPIYFHTQ |
| | (SEQ ID NO: 1) |
| the first | ATGGAAGTGCAGTTAGGGCTGGGAAGGGTCTACCCTCGGCCGCCG |
| GOI | TCCAAGACCTACCGAGGAGCTTTCCAGAATCTGTTCCAGAGCGTGC |
| | GCGAAGTGATCCAGAACCCGGGCCCCAGGCACCCAGAGGCCGCG |
| | AGCGCAGCACCTCCCGGCGCCAGTTTGCTGCTGCTGCAGCAGCA |
| | GCAGCAGCAGCAGCAGCAGCAGCAGCAGCAGCAGCAGCAGCAGC |
| | AGCAGCAGCAGCAGCAAGAGACTAGCCCCAGGCAGCAGCAGCAG |
| | CAGCAGGGTGAGGATGGTTCTCCCCAAGCCCATCGTAGAGGCCCC |
| | ACAGGCTACCTGGTCCTGGATGAGGAACAGCAACCTTCACAGCCG |
| | CAGTCGGCCCTGGAGTGCCACCCCGAGAGAGGTTGCGTCCCAGA |
| | GCCTGGAGCCGCCGTGGCCGCCAGCAAGGGGCTGCCGCAGCAG |
| | CTGCCAGCACCTCCGGACGAGGATGACTCAGCTGCCCCATCCACG |
| | TTGTCCCTGCTGGGCCCCACTTTCCCCGGCTTAAGCAGCTGCTCC |
| | GCTGACCTTAAAGACATCCTGAGCGAGGCCAGCACCATGCAACTC |
| | CTTCAGCAACAGCAGCAGGAAGCAGTATCCGAAGGCAGCAGCAGC |
| | GGGAGAGCGAGGGAGGCCTCGGGGGCTCCCACTTCCTCCAAGGA |
| | CAATTACTTAGGGGGCACTTCGACCATTTCTGACAACGCCAAGGAG |
| | TTGTGTAAGGCAGTGTCGGTGTCCATGGGCCTGGGTGTGGAGGCG |
| | TTGGAGCATCTGAGTCCAGGGGAACAGCTTCGGGGGGATTGCATG |
| | TACGCCCCACTTTTGGGAGTTCCACCCGCTGTGCGTCCCACTCCTT |
| | GTGCCCCATTGGCCGAATGCAAAGGTTCTCTGCTAGACGACAGCG |
| | CAGGCAAGAGCACTGAAGATACTGCTGAGTATTCCCCTTTCAAGGG |
| | AGGTTACACCAAAGGGCTAGAAGGCGAGAGCCTAGGCTGCTCTGG |
| | CAGCGCTGCAGCAGGGAGCTCCGGGACACTTGAACTGCCGTCTAC |
| | CCTGTCTCTCTACAAGTCCGGAGCACTGGACGAGGCAGCTGCGTA |
| | CCAGAGTCGCGACTACTACAACTTTCCACTGGCTCTGGCCGGACC |
| | GCCGCCCCCTCCGCCGCCTCCCCATCCCCACGCTCGCATCAAGCT |
| | GGAGAACCCGCTGGACTACGGCAGCGCCTGGGCGGCTGCGGCGG |
| | CGCAGTGCCGCTATGGGGACCTGGCGAGCCTGCATGGCGCGGGT |
| | GCAGCGGGACCCGGTTCTGGGTCACCCTCAGCCGCCGCTTCCTC |
| | ATCCTGGCACACTCTCTTCACAGCCGAAGAAGGCCAGTTGTATGGA |
| | CCGTGTGGTGGTGGTGGGGGTGGTGGCGGCGGCGGCGGCGGCG |
| | GCGGCGGCGGCGGCGGCGGCGGCGGCGGCGGCGAGGCGGGAG |
| | CTGTAGCCCCCTACGGCTACACTCGGCCCCCTCAGGGGCTGGCGG |
| | GCCAGGAAAGCGACTTCACCGCACCTGATGTGTGGTACCCTGGCG |
| | GCATGGTGAGCAGAGTGCCCTATCCCAGTCCCACTTGTGTCAAAAG |
| | CGAAATGGGCCCCTGGATGGATAGCTACTCCGGACCTTACGGGGA |
| | CATGCGTTTGGAGACTGCCAGGGACCATGTTTTGCCCATTGACTAT |
| | TACTTTCCACCCCAGAAGACCTGCCTGATCTGTGGAGATGAAGCTT |
| | CTGGGTGTCACTATGGAGCTCTCACATGTGGAAGCTGCAAGGTCTT |
| | CTTCAAAAGAGCCGCTGAAGGGAAACAGAAGTACCTGTGCGCCAG |
| | CAGAAATGATTGCACTATTGATAAATTCCGAAGGAAAAATTGTCCATC |
| | TTGTCGTCTTCGGAAATGTTATGAAGCAGGGATGACTCTGGGAGCC |
| | CGGAAGCTGAAGAAACTTGGTAATCTGAAACTACAGGAGGAAGGA |
| | GAGGCTTCCAGCACCACCAGCCCCACTGAGGAGACAACCCAGAAG |
| | CTGACAGTGTCACACATTGAAGGCTATGAATGTCAGCCCATCTTTCT |
| | GAATGTCCTGGAAGCCATTGAGCCAGGTGTAGTGTGTGCTGGACA |
| | CGACAACAACCAGCCCGACTCCTTTGCAGCCTTGCTCTCTAGCCTC |
| | AATGAACTGGGAGAGAGACAGCTTGTACACGTGGTCAAGTGGGCC |
| | AAGGCCTTGCCTGGCTTCCGCAACTTACACGTGGACGACCAGATG |
| | GCTGTCATTCAGTACTCCTGGATGGGGCTCATGGTGTTTGCCATGG |
| | GCTGGCGATCCTTCACCAATGTCAACTCCAGGATGCTCTACTTCGC |
| | CCCTGATCTGGTTTTCAATGAGTACCGCATGCACAAGTCCCGGATG |
| | TACAGCCAGTGTGTCCGAATGAGGCACCTCTCTCAAGAGTTTGGAT |
| | GGCTCCAAATCACCCCCCAGGAATTCCTGTGCATGAAAGCACTGCT |
| | ACTCTTCAGCATTATTCCAGTGGATGGGCTGAAAAATCAAAAATTCT |
| | TTGATGAACTTCGAATGAACTACATCAAGGAACTCGATCGTATCATT |
| | GCATGCAAAAGAAAAAATCCCACATCCTGCTCAAGACGCTTCTACC |
| | AGCTCACCAAGCTCCTGGACTCCGTGCAGCCTATTGCGAGAGAGC |
| | TGCATCAGTTCACTTTTGACCTGCTAATCAAGTCACACATGGTGAGC |
| | GTGGACTTTCCGGAAATGATGGCAGAGATCATCTCTGTGCAAGTGC |
| | CCAAGATCCTTTCTGGGAAAGTCAAGCCCATCTATTTCCACACCCA |
| | GTGA (SEQ ID NO: 2) |

Cell lines were transfected with the expression plasmids and expression level of the first GOI was detected by qRT-PCR. FIG. 2 is a schematic diagram illustrating relative target gene (i.e., the first GOI) expressions of the prostate cancer cells (22Rv1) transfected with the expression plasmids according to the present disclosure. As shown in FIG. 2, in the prostate cancer cells (22Rv1) transfected with plasmid 0001 and plasmid 0002, the expression level of the exogenous androgen receptor mRNAs encoded by the first GOI is significantly elevated driven by the expression cassette under the alternative PGC-1a promoter.

### Example 3: Design Adeno-Associated Viruses (AAVs)

Adeno-associated viruses (AAVs) were designed and constructed based on the expression plasmids.

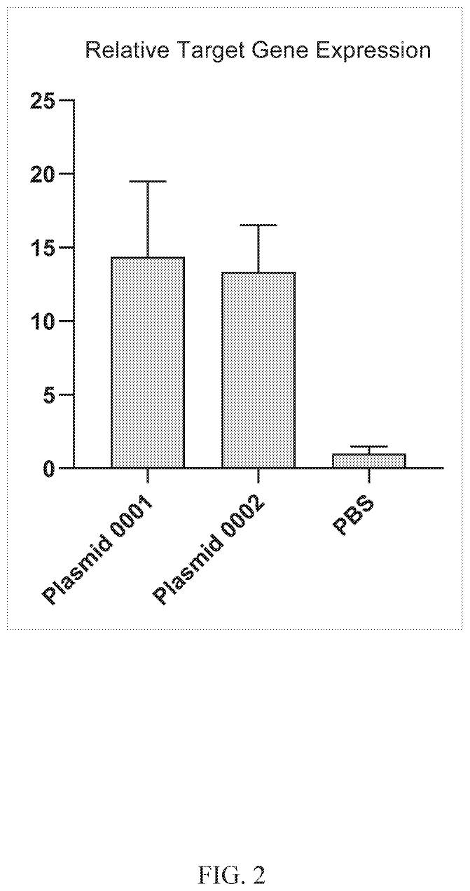

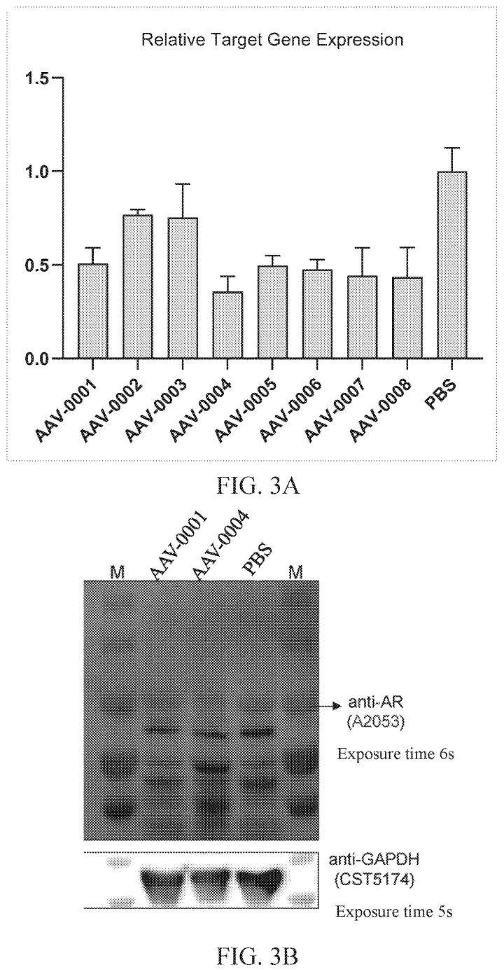

Non-human primates (NHPs) were infected with the AAVs and expression level of the gene of interest from a tissue of concerned was detected by qRT-PCR 90 days post-infection. In some embodiments, muscle tissues of the NHPs infected with the AAVs encoding different versions of the expression cassettes for various genes of interest and artificial miRNAs were analyzed. FIG. 3A is a schematic diagram illustrating relative target gene expressions of the NHPs infected with the AAVs according to the present disclosure. AAV-0001, AAV-0004, AAV-0005, AAV-0006, AAV-0007, and AAV-0008 effectively reduced the mRNA expression level of endogenous gene of interest (i.e., the second GOI) in the muscle tissue 90 days post-infection. The constructed sequences are listed in Table 3.

TABLE 3
Sequences of AAVs

| | | SEQ ID |
|---|---|---|
| Description | Sequence | NO: |
| AAV-0001 | TGGAGAACAGCCTGTTCTCCACACGTGTAATCCCGTGGGAGG | 40 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAAGAGACTAGCCCCAGGCAGCAGCAGCAGCAG | |
| | CAGGGTGAGGATGGTTCTCCCCAAGCCCATCGTAGAGGCCC | |
| | CACAGGCTACCTGGTCCTGGATGAGGAACAGCAACCTTCACA | |
| | GCCGCAGTCGGCCCTGGAGTGCCACCCCGAGAGAGGTTGC | |
| | GTCCCAGAGCCTGGAGCCGCCGTGGCCGCCAGCAAGGGGC | |
| | TGCCGCAGCAGCTGCCAGCACCTCCGGACGAGGATGACTCA | |
| | GCTGCCCCATCCACGTTGTCCCTGCTGGGCCCCACTTTCCCC | |
| | GGCTTAAGCAGCTGCTCCGCTGACCTTAAAGACATCCTGAGC | |
| | GAGGCCAGCACCATGCAACTCCTTCAGCAACAGCAGCAGGA | |
| | AGCAGTATCCGAAGGCAGCAGCAGCGGGAGAGCGAGGGAG | |
| | GCCTCGGGGGCTCCCACTTCCTCCAAGGACAATTACTTAGGG | |
| | GGCACTTCGACCATTTCTGACAACGCCAAGGAGTTGTGTAAG | |
| | GCAGTGTCGGTGTCCATGGGCCTGGGTGTGGAGGCGTTGGA | |
| | GCATCTGAGTCCAGGGGAACAGCTTCGGGGGGATTGCATGTA | |
| | CGCCCCACTTTTGGGAGTTCCACCCGCTGTGCGTCCCACTC | |
| | CTTGTGCCCCATTGGCCGAATGCAAAGGTTCTCTGCTAGACG | |
| | ACAGCGCAGGCAAGAGCACTGAAGATACTGCTGAGTATTCCC | |
| | CTTTCAAGGGAGGTTACACCAAAGGGCTAGAAGGCGAGAGC | |
| | CTAGGCTGCTCTGGCAGCGCTGCAGCAGGGAGCTCCGGGAC | |
| | ACTTGAACTGCCGTCTACCCTGTCTCTCTACAAGTCCGGAGC | |
| | ACTGGACGAGGCAGCTGCGTACCAGAGTCGCGACTACTACAA | |
| | CTTTCCACTGGCTCTGGCCGGACCGCCGCCCCCTCCGCCGC | |
| | CTCCCCATCCCCACGCTCGCATCAAGCTGGAGAACCCGCTG | |
| | GACTACGGCAGCGCCTGGGCGGCTGCGGCGGCGCAGTGCC | |
| | GCTATGGGGACCTGGCGAGCCTGCATGGCGCGGGTGCAGC | |
| | GGGACCCGGTTCTGGGTCACCCTCAGCCGCCGCTTCCTCAT | |
| | CCTGGCACACTCTCTTCACAGCCGAAGAAGGCCAGTTGTATG | |
| | GACCGTGTGGTGGTGGTGGGGGTGGTGGCGGCGGCGGCG | |
| | GCGGCGGCGGCGGCGGCGGCGGCGGCGGCGGCGGCGGC | |
| | GAGGCGGGAGCTGTAGCCCCCTACGGCTACACTCGGCCCCC | |
| | TCAGGGGCTGGCGGGCCAGGAAAGCGACTTCACCGCACCTG | |
| | ATGTGTGGTACCCTGGCGGCATGGTGAGCAGAGTGCCCTATC | |
| | CCAGTCCCACTTGTGTCAAAAGCGAAATGGGCCCCTGGATGG | |
| | ATAGCTACTCCGGACCTTACGGGGACATGCGTTTGGAGACTG | |
| | CCAGGGACCATGTTTTGCCCATTGACTATTACTTTCCACCCCA | |
| | GAAGACCTGCCTGATCTGTGGAGATGAAGCTTCTGGGTGTCA | |
| | CTATGGAGCTCTCACATGTGGAAGCTGCAAGGTCTTCTTCAAA | |
| | AGAGCCGCTGAAGGGAAACAGAAGTACCTGTGCGCCAGCAG | |
| | AAATGATTGCACTATTGATAAATTCCGAAGGAAAAATTGTCCAT | |
| | CTTGTCGTCTTCGGAAATGTTATGAAGCAGGGATGACTCTGG | |
| | GAGCCCGGAAGCTGAAGAAACTTGGTAATCTGAAACTACAGG | |
| | AGGAAGGAGAGGCTTCCAGCACCACCAGCCCCACTGAGGAG | |
| | ACAACCCAGAAGCTGACAGTGTCACACATTGAAGGCTATGAA | |
| | TGTCAGCCCATCTTTCTGAATGTCCTGGAAGCCATTGAGCCA | |
| | GGTGTAGTGTGTGCTGGACACGACAACAACCAGCCCGACTC | |
| | CTTTGCAGCCTTGCTCTCTAGCCTCAATGAACTGGGAGAGAG | |
| | ACAGCTTGTACACGTGGTCAAGTGGGCCAAGGCCTTGCCTG | |
| | GCTTCCGCAACTTACACGTGGACGACCAGATGGCTGTCATTC | |
| | AGTACTCCTGGATGGGGCTCATGGTGTTTGCCATGGGCTGGC | |
| | GATCCTTCACCAATGTCAACTCCAGGATGCTCTACTTCGCCCC | |
| | TGATCTGGTTTTCAATGAGTACCGCATGCACAAGTCCCGGATG | |
| | TACAGCCAGTGTGTCCGAATGAGGCACCTCTCTCAAGAGTTT | |
| | GGATGGCTCCAAATCACCCCCCAGGAATTCCTGTGCATGAAA | |
| | GCACTGCTACTCTTCAGCATTATTCCAGTGGATGGGCTGAAAA | |
| | ATCAAAAATTCTTTGATGAACTTCGAATGAACTACATCAAGGAA | |
| | CTCGATCGTATCATTGCATGCAAAAGAAAAAATCCCACATCCT | |
| | GCTCAAGACGCTTCTACCAGCTCACCAAGCTCCTGGACTCCG | |
| | TGCAGCCTATTGCGAGAGAGCTGCATCAGTTCACTTTTGACCT | |
| | GCTAATCAAGTCACACATGGTGAGCGTGGACTTTCCGGAAAT | |
| | GATGGCAGAGATCATCTCTGTGCAAGTGCCCAAGATCCTTTCT | |
| | GGGAAAGTCAAGCCCATCTATTTCCACACCCAGTGAAGGTAA | |
| | GTCTGGAGGCTTGCTTTGGGCTGTATGCTGGAATCTTCCACC | |
| | TACTTCCCTTTTTGGCCTCTGACTGAAGGGAGTATTGGAAGAT | |
| | TCCAGGACACAAGGCCCTTATCAGCACTCTGGAGGCTTGCTT | |
| | TGGGCTGTATGCTGTTATAACAGGCAGAAGACATCTTTTGGCC | |
| | TCTGACTGAGATGTCTCTTCTGTTATAACAGGACACAAGGCCC | |
| | TTATCAGCACTCTGGAGGCTTGCTTTGGGCTGTATGCTGTTTA | |
| | AAGGCATACAACACCATTTTTGGCCTCTGACTGAATGGTGTGT | |
| | TGCCTTTAAACAGGACACAAGGCCCTTATCAGCACTCTCTCTC | |
| | TCTGCAGGGTATTTGTGAAATTTGTGATGCTATTGCTTTATTTG | |
| | TAACCATCTAGCTTTATTTGTGAAATTTGTGATGCTATTGCTTTA | |
| | TTTGTAACCATTATAAGCTGCAATAAACAAGTTAACAACAACAA | |
| | TTGCATTCATTTTATGTTTCAGGTTCAGGGGGAGATGTGGGAG | |
| | GTTTTTTAAAGCGG | |
| AAV-0002 | TGGAGAACAGCCTGTTCTCCACACGTGTAATCCCGTGGGAGG | 41 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAAGAGACTAGCCCCAGGCAGCAGCAGCAGCAG | |
| | CAGGGTGAGGATGGTTCTCCCCAAGCCCATCGTAGAGGCCC | |
| | CACAGGCTACCTGGTCCTGGATGAGGAACAGCAACCTTCACA | |
| | GCCGCAGTCGGCCCTGGAGTGCCACCCCGAGAGAGGTTGC | |
| | GTCCCAGAGCCTGGAGCCGCCGTGGCCGCCAGCAAGGGGC | |
| | TGCCGCAGCAGCTGCCAGCACCTCCGGACGAGGATGACTCA | |
| | GCTGCCCCATCCACGTTGTCCCTGCTGGGCCCCACTTTCCCC | |
| | GGCTTAAGCAGCTGCTCCGCTGACCTTAAAGACATCCTGAGC | |
| | GAGGCCAGCACCATGCAACTCCTTCAGCAACAGCAGCAGGA | |
| | AGCAGTATCCGAAGGCAGCAGCAGCGGGAGAGCGAGGGAG | |
| | GCCTCGGGGGCTCCCACTTCCTCCAAGGACAATTACTTAGGG | |
| | GGCACTTCGACCATTTCTGACAACGCCAAGGAGTTGTGTAAG | |
| | GCAGTGTCGGTGTCCATGGGCCTGGGTGTGGAGGCGTTGGA | |
| | GCATCTGAGTCCAGGGGAACAGCTTCGGGGGGATTGCATGTA | |
| | CGCCCCACTTTTGGGAGTTCCACCCGCTGTGCGTCCCACTC | |
| | CTTGTGCCCCATTGGCCGAATGCAAAGGTTCTCTGCTAGACG | |
| | ACAGCGCAGGCAAGAGCACTGAAGATACTGCTGAGTATTCCC | |
| | CTTTCAAGGGAGGTTACACCAAAGGGCTAGAAGGCGAGAGC | |
| | CTAGGCTGCTCTGGCAGCGCTGCAGCAGGGAGCTCCGGGAC | |
| | ACTTGAACTGCCGTCTACCCTGTCTCTCTACAAGTCCGGAGC | |
| | ACTGGACGAGGCAGCTGCGTACCAGAGTCGCGACTACTACAA | |
| | CTTTCCACTGGCTCTGGCCGGACCGCCGCCCCCTCCGCCGC | |
| | CTCCCCATCCCCACGCTCGCATCAAGCTGGAGAACCCGCTG | |
| | GACTACGGCAGCGCCTGGGCGGCTGCGGCGGCGCAGTGCC | |
| | GCTATGGGGACCTGGCGAGCCTGCATGGCGCGGGTGCAGC | |
| | GGGACCCGGTTCTGGGTCACCCTCAGCCGCCGCTTCCTCAT | |
| | CCTGGCACACTCTCTTCACAGCCGAAGAAGGCCAGTTGTATG | |
| | GACCGTGTGGTGGTGGTGGGGGTGGTGGCGGCGGCGGCG | |
| | GCGGCGGCGGCGGCGGCGGCGGCGGCGGCGGCGGCGGC | |
| | GAGGCGGGAGCTGTAGCCCCCTACGGCTACACTCGGCCCCC | |
| | TCAGGGGCTGGCGGGCCAGGAAAGCGACTTCACCGCACCTG | |
| | ATGTGTGGTACCCTGGCGGCATGGTGAGCAGAGTGCCCTATC | |
| | CCAGTCCCACTTGTGTCAAAAGCGAAATGGGCCCCTGGATGG | |
| | ATAGCTACTCCGGACCTTACGGGGACATGCGTTTGGAGACTG | |
| | CCAGGGACCATGTTTTGCCCATTGACTATTACTTTCCACCCCA | |
| | GAAGACCTGCCTGATCTGTGGAGATGAAGCTTCTGGGTGTCA | |
| | CTATGGAGCTCTCACATGTGGAAGCTGCAAGGTCTTCTTCAAA | |
| | AGAGCCGCTGAAGGGAAACAGAAGTACCTGTGCGCCAGCAG | |
| | AAATGATTGCACTATTGATAAATTCCGAAGGAAAAATTGTCCAT | |
| | CTTGTCGTCTTCGGAAATGTTATGAAGCAGGGATGACTCTGG | |
| | GAGCCCGGAAGCTGAAGAAACTTGGTAATCTGAAACTACAGG | |
| | AGGAAGGAGAGGCTTCCAGCACCACCAGCCCCACTGAGGAG | |
| | ACAACCCAGAAGCTGACAGTGTCACACATTGAAGGCTATGAA | |
| | TGTCAGCCCATCTTTCTGAATGTCCTGGAAGCCATTGAGCCA | |
| | GGTGTAGTGTGTGCTGGACACGACAACAACCAGCCCGACTC | |
| | CTTTGCAGCCTTGCTCTCTAGCCTCAATGAACTGGGAGAGAG | |
| | ACAGCTTGTACACGTGGTCAAGTGGGCCAAGGCCTTGCCTG | |
| | GCTTCCGCAACTTACACGTGGACGACCAGATGGCTGTCATTC | |
| | AGTACTCCTGGATGGGGCTCATGGTGTTTGCCATGGGCTGGC | |
| | GATCCTTCACCAATGTCAACTCCAGGATGCTCTACTTCGCCCC | |
| | TGATCTGGTTTTCAATGAGTACCGCATGCACAAGTCCCGGATG | |
| | TACAGCCAGTGTGTCCGAATGAGGCACCTCTCTCAAGAGTTT | |
| | GGATGGCTCCAAATCACCCCCCAGGAATTCCTGTGCATGAAA | |
| | GCACTGCTACTCTTCAGCATTATTCCAGTGGATGGGCTGAAAA | |
| | ATCAAAAATTCTTTGATGAACTTCGAATGAACTACATCAAGGAA | |
| | CTCGATCGTATCATTGCATGCAAAAGAAAAAATCCCACATCCT | |
| | GCTCAAGACGCTTCTACCAGCTCACCAAGCTCCTGGACTCCG | |
| | TGCAGCCTATTGCGAGAGAGCTGCATCAGTTCACTTTTGACCT | |
| | GCTAATCAAGTCACACATGGTGAGCGTGGACTTTCCGGAAAT | |
| | GATGGCAGAGATCATCTCTGTGCAAGTGCCCAAGATCCTTTCT | |
| | GGGAAAGTCAAGCCCATCTATTTCCACACCCAGTGACTGTGC | |
| | CTTCTAGTTGCCAGCCATCTGTTGTTTGCCCCTCCCCCGTGC | |
| | CTTCCTTGACCCTGGAAGGTGCCACTCCCACTGTCCTTTCCT | |
| | AATAAAATGAGGAAATTGCATCGCATTGTCTGAGTAGGTGTCA | |
| | TTCTATTCTGGGGGGTGGGGGGGGCAGGACAGCAAGGGG | |
| | GAGGATTGGGAAGAGAATAGCAGGCATGCTGGGGACTGGAG | |
| | GCTTGCTTTGGGCTGTATGCTGGAATCTTCCACCTACTTCCCT | |
| | TTTTGGCCTCTGACTGAAGGGAGTATTGGAAGATTCCAGGAC | |
| | ACAAGGCCCTTATCAGCACTCTGGAGGCTTGCTTTGGGCTGT | |
| | ATGCTGTTATAACAGGCAGAAGACATCTTTTGGCCTCTGACTG | |
| | AGATGTCTCTTCTGTTATAACAGGACACAAGGCCCTTATCAGC | |
| | ACTCTGGAGGCTTGCTTTGGGCTGTATGCTGTTTAAAGGCATA | |
| | CAACACCATTTTTGGCCTCTGACTGAATGGTGTGTTGCCTTTA | |
| | AACAGGACACAAGGCCCTTATCAGCACTTATTTGTGAAATTTG | |
| | TGATGCTATTGCTTTATTTGTAACCATCTAGCTTTATTTGTGAAA | |
| | TTTGTGATGCTATTGCTTTATTTGTAACCATTATAAGCTGCAATA | |
| | AACAAGTTAACAACAACAATTGCATTCATTTTATGTTTCAGGTT | |
| | CAGGGGGAGATGTGGGAGGTTTTTTAAAGCGG | |
| AAV-0003 | TGGAGAACAGCCTGTTCTCCACACGTGTAATCCCGTGGGAGG | 42 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAGCAGCAGCAGCAGCAGCAGCAGCAGCAGCA | |
| | GCAGCAGCAGCAGCAGCAGCAGCAGCAGCAGCAGCAGCAG | |
| | CAGCAAGAGACTAGCCCCAGGCAGCAGCAGCAGCAGCAGG | |
| | GTGAGGATGGTTCTCCCCAAGCCCATCGTAGAGGCCCCACA | |
| | GGCTACCTGGTCCTGGATGAGGAACAGCAACCTTCACAGCC | |
| | GCAGTCGGCCCTGGAGTGCCACCCCGAGAGAGGTTGCGTCC | |
| | CAGAGCCTGGAGCCGCCGTGGCCGCCAGCAAGGGGCTGCC | |
| | GCAGCAGCTGCCAGCACCTCCGGACGAGGATGACTCAGCTG | |
| | CCCCATCCACGTTGTCCCTGCTGGGCCCCACTTTCCCCGGCT | |
| | TAAGCAGCTGCTCCGCTGACCTTAAAGACATCCTGAGCGAGG | |
| | CCAGCACCATGCAACTCCTTCAGCAACAGCAGCAGGAAGCA | |
| | GTATCCGAAGGCAGCAGCAGCGGGAGAGCGAGGGAGGCCT | |
| | CGGGGGCTCCCACTTCCTCCAAGGACAATTACTTAGGGGGCA | |
| | CTTCGACCATTTCTGACAACGCCAAGGAGTTGTGTAAGGCAG | |
| | TGTCGGTGTCCATGGGCCTGGGTGTGGAGGCGTTGGAGCAT | |
| | CTGAGTCCAGGGGAACAGCTTCGGGGGGATTGCATGTACGC | |
| | CCCACTTTTGGGAGTTCCACCCGCTGTGCGTCCCACTCCTTG | |
| | TGCCCCATTGGCCGAATGCAAAGGTTCTCTGCTAGACGACAG | |
| | CGCAGGCAAGAGCACTGAAGATACTGCTGAGTATTCCCCTTT | |
| | CAAGGGAGGTTACACCAAAGGGCTAGAAGGCGAGAGCCTAG | |
| | GCTGCTCTGGCAGCGCTGCAGCAGGGAGCTCCGGGACACTT | |
| | GAACTGCCGTCTACCCTGTCTCTCTACAAGTCCGGAGCACTG | |
| | GACGAGGCAGCTGCGTACCAGAGTCGCGACTACTACAACTTT | |
| | CCACTGGCTCTGGCCGGACCGCCGCCCCCTCCGCCGCCTC | |
| | CCCATCCCCACGCTCGCATCAAGCTGGAGAACCCGCTGGAC | |
| | TACGGCAGCGCCTGGGCGGCTGCGGCGGCGCAGTGCCGCT | |
| | ATGGGGACCTGGCGAGCCTGCATGGCGCGGGTGCAGCGGG | |
| | ACCCGGTTCTGGGTCACCCTCAGCCGCCGCTTCCTCATCCTG | |
| | GCACACTCTCTTCACAGCCGAAGAAGGCCAGTTGTATGGACC | |
| | GTGTGGTGGTGGTGGGGGTGGTGGCGGCGGCGGCGGCGG | |
| | CGGCGGCGGCGGCGGCGGCGGCGGCGGCGGCGGCGAGG | |
| | CGGGAGCTGTAGCCCCCTACGGCTACACTCGGCCCCCTCAG | |
| | GGGCTGGCGGGCCAGGAAAGCGACTTCACCGCACCTGATGT | |
| | GTGGTACCCTGGCGGCATGGTGAGCAGAGTGCCCTATCCCA | |
| | GTCCCACTTGTGTCAAAAGCGAAATGGGCCCCTGGATGGATA | |
| | GCTACTCCGGACCTTACGGGGACATGCGTTTGGAGACTGCCA | |
| | GGGACCATGTTTTGCCCATTGACTATTACTTTCCACCCCAGAA | |
| | GACCTGCCTGATCTGTGGAGATGAAGCTTCTGGGTGTCACTA | |
| | TGGAGCTCTCACATGTGGAAGCTGCAAGGTCTTCTTCAAAAG | |
| | AGCCGCTGAAGGGAAACAGAAGTACCTGTGCGCCAGCAGAA | |
| | ATGATTGCACTATTGATAAATTCCGAAGGAAAAATTGTCCATCT | |
| | TGTCGTCTTCGGAAATGTTATGAAGCAGGGATGACTCTGGGA | |
| | GCCCGGAAGCTGAAGAAACTTGGTAATCTGAAACTACAGGAG | |
| | GAAGGAGAGGCTTCCAGCACCACCAGCCCCACTGAGGAGAC | |
| | AACCCAGAAGCTGACAGTGTCACACATTGAAGGCTATGAATG | |
| | TCAGCCCATCTTTCTGAATGTCCTGGAAGCCATTGAGCCAGG | |
| | TGTAGTGTGTGCTGGACACGACAACAACCAGCCCGACTCCTT | |
| | TGCAGCCTTGCTCTCTAGCCTCAATGAACTGGGAGAGAGACA | |
| | GCTTGTACACGTGGTCAAGTGGGCCAAGGCCTTGCCTGGCT | |
| | TCCGCAACTTACACGTGGACGACCAGATGGCTGTCATTCAGT | |
| | ACTCCTGGATGGGGCTCATGGTGTTTGCCATGGGCTGGCGAT | |
| | CCTTCACCAATGTCAACTCCAGGATGCTCTACTTCGCCCCTGA | |
| | TCTGGTTTTCAATGAGTACCGCATGCACAAGTCCCGGATGTAC | |
| | AGCCAGTGTGTCCGAATGAGGCACCTCTCTCAAGAGTTTGGA | |
| | TGGCTCCAAATCACCCCCCAGGAATTCCTGTGCATGAAAGCA | |
| | CTGCTACTCTTCAGCATTATTCCAGTGGATGGGCTGAAAAATC | |
| | AAAAATTCTTTGATGAACTTCGAATGAACTACATCAAGGAACTC | |
| | GATCGTATCATTGCATGCAAAAGAAAAAATCCCACATCCTGCT | |
| | CAAGACGCTTCTACCAGCTCACCAAGCTCCTGGACTCCGTGC | |
| | AGCCTATTGCGAGAGAGCTGCATCAGTTCACTTTTGACCTGCT | |
| | AATCAAGTCACACATGGTGAGCGTGGACTTTCCGGAAATGAT | |
| | GGCAGAGATCATCTCTGTGCAAGTGCCCAAGATCCTTTCTGG | |
| | GAAAGTCAAGCCCATCTATTTCCACACCCAGTGACTGTGCCTT | |
| | CTAGTTGCCAGCCATCTGTTGTTTGCCCCTCCCCCGTGCCTT | |
| | CCTTGACCCTGGAAGGTGCCACTCCCACTGTCCTTTCCTAATA | |
| | AAATGAGGAAATTGCATCGCATTGTCTGAGTAGGTGTCATTCT | |
| | ATTCTGGGGGGTGGGGTGGGGCAGGACAGCAAGGGGGAGG | |
| | ATTGGGAAGAGAATAGCAGGCATGCTGGGGACTGGAGGCTT | |
| | GCTTTGGGCTGTATGCTGGAATCTTCCACCTACTTCCCTTTTT | |
| | GGCCTCTGACTGAAGGGAGTATTGGAAGATTCCAGGACACAA | |
| | GGCCCTTATCAGCACTCTGGAGGCTTGCTTTGGGCTGTATGC | |
| | TGTTATAACAGGCAGAAGACATCTTTTGGCCTCTGACTGAGAT | |
| | GTCTCTTCTGTTATAACAGGACACAAGGCCCTTATCAGCACTC | |
| | TGGAGGCTTGCTTTGGGCTGTATGCTGTTTAAAGGCATACAAC | |
| | ACCATTTTTGGCCTCTGACTGAATGGTGTGTTGCCTTTAAACA | |
| | GGACACAAGGCCCTTATCAGCACTTATTTGTGAAATTTGTGAT | |
| | GCTATTGCTTTATTTGTAACCATCTAGCTTTATTTGTGAAATTTG | |
| | TGATGCTATTGCTTTATTTGTAACCATTATAAGCTGCAATAAACA | |
| | AGTTAACAACAACAATTGCATTCATTTTATGTTTCAGGTTCAGG | |
| | GGGAGATGTGGGAGGTTTTTTAAAGCGG | |
| AAV-0004 | TGGAGAACAGCCTGTTCTCCACACGTGTAATCCCGTGGGAGG | 43 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAGCAGCAGCAGCAGCAGCAGCAGCAGCAGCA | |
| | GCAGCAGCAGCAGCAGCAGCAGCAGCAGCAGCAGCAGCAG | |
| | CAGCAAGAGACTAGCCCCAGGCAGCAGCAGCAGCAGCAGG | |
| | GTGAGGATGGTTCTCCCCAAGCCCATCGTAGAGGCCCCACA | |
| | GGCTACCTGGTCCTGGATGAGGAACAGCAACCTTCACAGCC | |
| | GCAGTCGGCCCTGGAGTGCCACCCCGAGAGAGGTTGCGTCC | |
| | CAGAGCCTGGAGCCGCCGTGGCCGCCAGCAAGGGGCTGCC | |
| | GCAGCAGCTGCCAGCACCTCCGGACGAGGATGACTCAGCTG | |
| | CCCCATCCACGTTGTCCCTGCTGGGCCCCACTTTCCCCGGCT | |
| | TAAGCAGCTGCTCCGCTGACCTTAAAGACATCCTGAGCGAGG | |
| | CCAGCACCATGCAACTCCTTCAGCAACAGCAGCAGGAAGCA | |
| | GTATCCGAAGGCAGCAGCAGCGGGAGAGCGAGGGAGGCCT | |
| | CGGGGGCTCCCACTTCCTCCAAGGACAATTACTTAGGGGGCA | |
| | CTTCGACCATTTCTGACAACGCCAAGGAGTTGTGTAAGGCAG | |
| | TGTCGGTGTCCATGGGCCTGGGTGTGGAGGCGTTGGAGCAT | |
| | CTGAGTCCAGGGGAACAGCTTCGGGGGGATTGCATGTACGC | |
| | CCCACTTTTGGGAGTTCCACCCGCTGTGCGTCCCACTCCTTG | |
| | TGCCCCATTGGCCGAATGCAAAGGTTCTCTGCTAGACGACAG | |
| | CGCAGGCAAGAGCACTGAAGATACTGCTGAGTATTCCCCTTT | |
| | CAAGGGAGGTTACACCAAAGGGCTAGAAGGCGAGAGCCTAG | |
| | GCTGCTCTGGCAGCGCTGCAGCAGGGAGCTCCGGGACACTT | |
| | GAACTGCCGTCTACCCTGTCTCTCTACAAGTCCGGAGCACTG | |
| | GACGAGGCAGCTGCGTACCAGAGTCGCGACTACTACAACTTT | |
| | CCACTGGCTCTGGCCGGACCGCCGCCCCCTCCGCCGCCTC | |
| | CCCATCCCCACGCTCGCATCAAGCTGGAGAACCCGCTGGAC | |
| | TACGGCAGCGCCTGGGCGGCTGCGGCGGCGCAGTGCCGCT | |
| | ATGGGGACCTGGCGAGCCTGCATGGCGCGGGTGCAGCGGG | |
| | ACCCGGTTCTGGGTCACCCTCAGCCGCCGCTTCCTCATCCTG | |
| | GCACACTCTCTTCACAGCCGAAGAAGGCCAGTTGTATGGACC | |
| | GTGTGGTGGTGGTGGGGGTGGTGGCGGCGGCGGCGGCGG | |
| | CGGCGGCGGCGGCGGCGGCGGCGGCGGCGGCGGCGAGG | |
| | CGGGAGCTGTAGCCCCCTACGGCTACACTCGGCCCCCTCAG | |
| | GGGCTGGCGGGCCAGGAAAGCGACTTCACCGCACCTGATGT | |
| | GTGGTACCCTGGCGGCATGGTGAGCAGAGTGCCCTATCCCA | |
| | GTCCCACTTGTGTCAAAAGCGAAATGGGCCCCTGGATGGATA | |
| | GCTACTCCGGACCTTACGGGGACATGCGTTTGGAGACTGCCA | |
| | GGGACCATGTTTTGCCCATTGACTATTACTTTCCACCCCAGAA | |
| | GACCTGCCTGATCTGTGGAGATGAAGCTTCTGGGTGTCACTA | |
| | TGGAGCTCTCACATGTGGAAGCTGCAAGGTCTTCTTCAAAAG | |
| | AGCCGCTGAAGGGAAACAGAAGTACCTGTGCGCCAGCAGAA | |
| | ATGATTGCACTATTGATAAATTCCGAAGGAAAAATTGTCCATCT | |
| | TGTCGTCTTCGGAAATGTTATGAAGCAGGGATGACTCTGGGA | |
| | GCCCGGAAGCTGAAGAAACTTGGTAATCTGAAACTACAGGAG | |
| | GAAGGAGAGGCTTCCAGCACCACCAGCCCCACTGAGGAGAC | |
| | AACCCAGAAGCTGACAGTGTCACACATTGAAGGCTATGAATG | |
| | TCAGCCCATCTTTCTGAATGTCCTGGAAGCCATTGAGCCAGG | |
| | TGTAGTGTGTGCTGGACACGACAACAACCAGCCCGACTCCTT | |
| | TGCAGCCTTGCTCTCTAGCCTCAATGAACTGGGAGAGAGACA | |
| | GCTTGTACACGTGGTCAAGTGGGCCAAGGCCTTGCCTGGCT | |
| | TCCGCAACTTACACGTGGACGACCAGATGGCTGTCATTCAGT | |
| | ACTCCTGGATGGGGCTCATGGTGTTTGCCATGGGCTGGCGAT | |
| | CCTTCACCAATGTCAACTCCAGGATGCTCTACTTCGCCCCTGA | |
| | TCTGGTTTTCAATGAGTACCGCATGCACAAGTCCCGGATGTAC | |
| | AGCCAGTGTGTCCGAATGAGGCACCTCTCTCAAGAGTTTGGA | |
| | TGGCTCCAAATCACCCCCCAGGAATTCCTGTGCATGAAAGCA | |
| | CTGCTACTCTTCAGCATTATTCCAGTGGATGGGCTGAAAAATC | |
| | AAAAATTCTTTGATGAACTTCGAATGAACTACATCAAGGAACTC | |
| | GATCGTATCATTGCATGCAAAAGAAAAAATCCCACATCCTGCT | |
| | CAAGACGCTTCTACCAGCTCACCAAGCTCCTGGACTCCGTGC | |
| | AGCCTATTGCGAGAGAGCTGCATCAGTTCACTTTTGACCTGCT | |
| | AATCAAGTCACACATGGTGAGCGTGGACTTTCCGGAAATGAT | |
| | GGCAGAGATCATCTCTGTGCAAGTGCCCAAGATCCTTTCTGG | |
| | GAAAGTCAAGCCCATCTATTTCCACACCCAGTGAAGGTAAGTC | |
| | TGGAGGCTTGCTTTGGGCTGTATGCTGGAATCTTCCACCTACT | |
| | TCCCTTTTTGGCCTCTGACTGAAGGGAGTATTGGAAGATTCCA | |
| | GGACACAAGGCCCTTATCAGCACTCTGGAGGCTTGCTTTGGG | |
| | CTGTATGCTGTTATAACAGGCAGAAGACATCTTTTGGCCTCTG | |
| | ACTGAGATGTCTCTTCTGTTATAACAGGACACAAGGCCCTTAT | |
| | CAGCACTCTGGAGGCTTGCTTTGGGCTGTATGCTGTTTAAAG | |
| | GCATACAACACCATTTTTGGCCTCTGACTGAATGGTGTGTTGC | |
| | CTTTAAACAGGACACAAGGCCCTTATCAGCACTCTCTCTCTCT | |
| | GCAGGGTATTTGTGAAATTTGTGATGCTATTGCTTTATTTGTAA | |
| | CCATCTAGCTTTATTTGTGAAATTTGTGATGCTATTGCTTTATTT | |
| | GTAACCATTATAAGCTGCAATAAACAAGTTAACAACAACAATTG | |
| | CATTCATTTTATGTTTCAGGTTCAGGGGGAGATGTGGGAGGTT | |
| | TTTTAAAGCGG | |
| AAV-0005 | TGGAGAACAGCCTGTTCTCCACACGTGTAATCCCGTGGGAGG | 44 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAGCAGCAGCAGCAGCAGCAGCAGCAGCAGCA | |
| | GCAGCAGCAGCAGCAGCAGCAGCAGCAGCAGCAGCAGCAG | |
| | CAGCAAGAGACTAGCCCCAGGCAGCAGCAGCAGCAGCAGG | |
| | GTGAGGATGGTTCTCCCCAAGCCCATCGTAGAGGCCCCACA | |
| | GGCTACCTGGTCCTGGATGAGGAACAGCAACCTTCACAGCC | |
| | GCAGTCGGCCCTGGAGTGCCACCCCGAGAGAGGTTGCGTCC | |
| | CAGAGCCTGGAGCCGCCGTGGCCGCCAGCAAGGGGCTGCC | |
| | GCAGCAGCTGCCAGCACCTCCGGACGAGGATGACTCAGCTG | |
| | CCCCATCCACGTTGTCCCTGCTGGGCCCCACTTTCCCCGGCT | |
| | TAAGCAGCTGCTCCGCTGACCTTAAAGACATCCTGAGCGAGG | |
| | CCAGCACCATGCAACTCCTTCAGCAACAGCAGCAGGAAGCA | |
| | GTATCCGAAGGCAGCAGCAGCGGGAGAGCGAGGGAGGCCT | |
| | CGGGGGCTCCCACTTCCTCCAAGGACAATTACTTAGGGGGCA | |
| | CTTCGACCATTTCTGACAACGCCAAGGAGTTGTGTAAGGCAG | |
| | TGTCGGTGTCCATGGGCCTGGGTGTGGAGGCGTTGGAGCAT | |
| | CTGAGTCCAGGGGAACAGCTTCGGGGGGATTGCATGTACGC | |
| | CCCACTTTTGGGAGTTCCACCCGCTGTGCGTCCCACTCCTTG | |
| | TGCCCCATTGGCCGAATGCAAAGGTTCTCTGCTAGACGACAG | |
| | CGCAGGCAAGAGCACTGAAGATACTGCTGAGTATTCCCCTTT | |
| | CAAGGGAGGTTACACCAAAGGGCTAGAAGGCGAGAGCCTAG | |
| | GCTGCTCTGGCAGCGCTGCAGCAGGGAGCTCCGGGACACTT | |
| | GAACTGCCGTCTACCCTGTCTCTCTACAAGTCCGGAGCACTG | |
| | GACGAGGCAGCTGCGTACCAGAGTCGCGACTACTACAACTTT | |
| | CCACTGGCTCTGGCCGGACCGCCGCCCCCTCCGCCGCCTC | |
| | CCCATCCCCACGCTCGCATCAAGCTGGAGAACCCGCTGGAC | |
| | TACGGCAGCGCCTGGGCGGCTGCGGCGGCGCAGTGCCGCT | |
| | ATGGGGACCTGGCGAGCCTGCATGGCGCGGGTGCAGCGGG | |
| | ACCCGGTTCTGGGTCACCCTCAGCCGCCGCTTCCTCATCCTG | |
| | GCACACTCTCTTCACAGCCGAAGAAGGCCAGTTGTATGGACC | |
| | GTGTGGTGGTGGTGGGGGTGGTGGCGGCGGCGGCGGCGG | |
| | CGGCGGCGGCGGCGGCGGCGGCGGCGGCGGCGGCGAGG | |
| | CGGGAGCTGTAGCCCCCTACGGCTACACTCGGCCCCCTCAG | |
| | GGGCTGGCGGGCCAGGAAAGCGACTTCACCGCACCTGATGT | |
| | GTGGTACCCTGGCGGCATGGTGAGCAGAGTGCCCTATCCCA | |
| | GTCCCACTTGTGTCAAAAGCGAAATGGGCCCCTGGATGGATA | |
| | GCTACTCCGGACCTTACGGGGACATGCGTTTGGAGACTGCCA | |
| | GGGACCATGTTTTGCCCATTGACTATTACTTTCCACCCCAGAA | |
| | GACCTGCCTGATCTGTGGAGATGAAGCTTCTGGGTGTCACTA | |
| | TGGAGCTCTCACATGTGGAAGCTGCAAGGTCTTCTTCAAAAG | |
| | AGCCGCTGAAGGGAAACAGAAGTACCTGTGCGCCAGCAGAA | |
| | ATGATTGCACTATTGATAAATTCCGAAGGAAAAATTGTCCATCT | |
| | TGTCGTCTTCGGAAATGTTATGAAGCAGGGATGACTCTGGGA | |
| | GCCCGGAAGCTGAAGAAACTTGGTAATCTGAAACTACAGGAG | |
| | GAAGGAGAGGCTTCCAGCACCACCAGCCCCACTGAGGAGAC | |
| | AACCCAGAAGCTGACAGTGTCACACATTGAAGGCTATGAATG | |
| | TCAGCCCATCTTTCTGAATGTCCTGGAAGCCATTGAGCCAGG | |
| | TGTAGTGTGTGCTGGACACGACAACAACCAGCCCGACTCCTT | |
| | TGCAGCCTTGCTCTCTAGCCTCAATGAACTGGGAGAGAGACA | |
| | GCTTGTACACGTGGTCAAGTGGGCCAAGGCCTTGCCTGGCT | |
| | TCCGCAACTTACACGTGGACGACCAGATGGCTGTCATTCAGT | |
| | ACTCCTGGATGGGGCTCATGGTGTTTGCCATGGGCTGGCGAT | |
| | CCTTCACCAATGTCAACTCCAGGATGCTCTACTTCGCCCCTGA | |
| | TCTGGTTTTCAATGAGTACCGCATGCACAAGTCCCGGATGTAC | |
| | AGCCAGTGTGTCCGAATGAGGCACCTCTCTCAAGAGTTTGGA | |
| | TGGCTCCAAATCACCCCCCAGGAATTCCTGTGCATGAAAGCA | |
| | CTGCTACTCTTCAGCATTATTCCAGTGGATGGGCTGAAAAATC | |
| | AAAAATTCTTTGATGAACTTCGAATGAACTACATCAAGGAACTC | |
| | GATCGTATCATTGCATGCAAAAGAAAAAATCCCACATCCTGCT | |
| | CAAGACGCTTCTACCAGCTCACCAAGCTCCTGGACTCCGTGC | |
| | AGCCTATTGCGAGAGAGCTGCATCAGTTCACTTTTGACCTGCT | |
| | AATCAAGTCACACATGGTGAGCGTGGACTTTCCGGAAATGAT | |
| | GGCAGAGATCATCTCTGTGCAAGTGCCCAAGATCCTTTCTGG | |
| | GAAAGTCAAGCCCATCTATTTCCACACCCAGTGAAGGTAAGTC | |
| | TGGAGGCTTGCTTTGGGCTGTATGCTGAATACTTGAAGGGTA | |
| | GATTCATTTTGGCCTCTGACTGATGAATCTACTTTCAATATTCA | |
| | GGACACAAGGCCCTTATCAGCACTCTGGAGGCTTGCTTTGGG | |
| | CTGTATGCTGTTATAACAGGCAGAAGACATCTTTTGGCCTCTG | |
| | ACTGAGATGTCTCTTCTGTTATAACAGGACACAAGGCCCTTAT | |
| | CAGCACTCTGGAGGCTTGCTTTGGGCTGTATGCTGTTTAAAG | |
| | GCATACAACACCATTTTTGGCCTCTGACTGAATGGTGTGTTGC | |
| | CTTTAAACAGGACACAAGGCCCTTATCAGCACTCTCTCTCTCT | |
| | GCAGGGTATTTGTGAAATTTGTGATGCTATTGCTTTATTTGTAA | |
| | CCATCTAGCTTTATTTGTGAAATTTGTGATGCTATTGCTTTATTT | |
| | GTAACCATTATAAGCTGCAATAAACAAGTTAACAACAACAATTG | |
| | CATTCATTTTATGTTTCAGGTTCAGGGGGAGATGTGGGAGGTT | |
| | TTTTAAAGCGG SALMIRSEDA GFVVITGVMS RRYLCMDFRG | |
| | NIFGSHYFDP ENCRFQHQTL ENGYDVYHSP QYHFLVSLGR | |
| | AKRAFLPGMN PPPYSQFLSR RNEIPLIHFN TPIPRRHTRS | |
| | AEDDSERDPLNVLKPRARMTPAPASCSQELPSAEDNSPMASDP | |
| | LGVVRGGRVNTHAGGTGPEGCRPFAKFI | |
| AAV-0006 | TGGAGAACAGCCTGTTCTCCACACGTGTAATCCCGTGGGAGG | 45 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAGCAGCAGCAGCAGCAGCAGCAGCAGCAGCA | |
| | GCAGCAGCAGCAGCAGCAGCAGCAGCAGCAGCAGCAGCAG | |
| | CAGCAAGAGACTAGCCCCAGGCAGCAGCAGCAGCAGCAGG | |
| | GTGAGGATGGTTCTCCCCAAGCCCATCGTAGAGGCCCCACA | |
| | GGCTACCTGGTCCTGGATGAGGAACAGCAACCTTCACAGCC | |
| | GCAGTCGGCCCTGGAGTGCCACCCCGAGAGAGGTTGCGTCC | |
| | CAGAGCCTGGAGCCGCCGTGGCCGCCAGCAAGGGGCTGCC | |
| | GCAGCAGCTGCCAGCACCTCCGGACGAGGATGACTCAGCTG | |
| | CCCCATCCACGTTGTCCCTGCTGGGCCCCACTTTCCCCGGCT | |
| | TAAGCAGCTGCTCCGCTGACCTTAAAGACATCCTGAGCGAGG | |
| | CCAGCACCATGCAACTCCTTCAGCAACAGCAGCAGGAAGCA | |
| | GTATCCGAAGGCAGCAGCAGCGGGAGAGCGAGGGAGGCCT | |
| | CGGGGGCTCCCACTTCCTCCAAGGACAATTACTTAGGGGGCA | |
| | CTTCGACCATTTCTGACAACGCCAAGGAGTTGTGTAAGGCAG | |
| | TGTCGGTGTCCATGGGCCTGGGTGTGGAGGCGTTGGAGCAT | |
| | CTGAGTCCAGGGGAACAGCTTCGGGGGGATTGCATGTACGC | |
| | CCCACTTTTGGGAGTTCCACCCGCTGTGCGTCCCACTCCTTG | |
| | TGCCCCATTGGCCGAATGCAAAGGTTCTCTGCTAGACGACAG | |
| | CGCAGGCAAGAGCACTGAAGATACTGCTGAGTATTCCCCTTT | |
| | CAAGGGAGGTTACACCAAAGGGCTAGAAGGCGAGAGCCTAG | |
| | GCTGCTCTGGCAGCGCTGCAGCAGGGAGCTCCGGGACACTT | |
| | GAACTGCCGTCTACCCTGTCTCTCTACAAGTCCGGAGCACTG | |
| | GACGAGGCAGCTGCGTACCAGAGTCGCGACTACTACAACTTT | |
| | CCACTGGCTCTGGCCGGACCGCCGCCCCCTCCGCCGCCTC | |
| | CCCATCCCCACGCTCGCATCAAGCTGGAGAACCCGCTGGAC | |
| | TACGGCAGCGCCTGGGCGGCTGCGGCGGCGCAGTGCCGCT | |
| | ATGGGGACCTGGCGAGCCTGCATGGCGCGGGTGCAGCGGG | |
| | ACCCGGTTCTGGGTCACCCTCAGCCGCCGCTTCCTCATCCTG | |
| | GCACACTCTCTTCACAGCCGAAGAAGGCCAGTTGTATGGACC | |
| | GTGTGGTGGTGGTGGGGGTGGTGGCGGCGGCGGCGGCGG | |
| | CGGCGGCGGCGGCGGCGGCGGCGGCGGCGGCGGCGAGG | |
| | CGGGAGCTGTAGCCCCCTACGGCTACACTCGGCCCCCTCAG | |
| | GGGCTGGCGGGCCAGGAAAGCGACTTCACCGCACCTGATGT | |
| | GTGGTACCCTGGCGGCATGGTGAGCAGAGTGCCCTATCCCA | |
| | GTCCCACTTGTGTCAAAAGCGAAATGGGCCCCTGGATGGATA | |
| | GCTACTCCGGACCTTACGGGGACATGCGTTTGGAGACTGCCA | |
| | GGGACCATGTTTTGCCCATTGACTATTACTTTCCACCCCAGAA | |
| | GACCTGCCTGATCTGTGGAGATGAAGCTTCTGGGTGTCACTA | |
| | TGGAGCTCTCACATGTGGAAGCTGCAAGGTCTTCTTCAAAAG | |
| | AGCCGCTGAAGGGAAACAGAAGTACCTGTGCGCCAGCAGAA | |
| | ATGATTGCACTATTGATAAATTCCGAAGGAAAAATTGTCCATCT | |
| | TGTCGTCTTCGGAAATGTTATGAAGCAGGGATGACTCTGGGA | |
| | GCCCGGAAGCTGAAGAAACTTGGTAATCTGAAACTACAGGAG | |
| | GAAGGAGAGGCTTCCAGCACCACCAGCCCCACTGAGGAGAC | |
| | AACCCAGAAGCTGACAGTGTCACACATTGAAGGCTATGAATG | |
| | TCAGCCCATCTTTCTGAATGTCCTGGAAGCCATTGAGCCAGG | |
| | TGTAGTGTGTGCTGGACACGACAACAACCAGCCCGACTCCTT | |
| | TGCAGCCTTGCTCTCTAGCCTCAATGAACTGGGAGAGAGACA | |
| | GCTTGTACACGTGGTCAAGTGGGCCAAGGCCTTGCCTGGCT | |
| | TCCGCAACTTACACGTGGACGACCAGATGGCTGTCATTCAGT | |
| | ACTCCTGGATGGGGCTCATGGTGTTTGCCATGGGCTGGCGAT | |
| | CCTTCACCAATGTCAACTCCAGGATGCTCTACTTCGCCCCTGA | |
| | TCTGGTTTTCAATGAGTACCGCATGCACAAGTCCCGGATGTAC | |
| | AGCCAGTGTGTCCGAATGAGGCACCTCTCTCAAGAGTTTGGA | |
| | TGGCTCCAAATCACCCCCCAGGAATTCCTGTGCATGAAAGCA | |
| | CTGCTACTCTTCAGCATTATTCCAGTGGATGGGCTGAAAAATC | |
| | AAAAATTCTTTGATGAACTTCGAATGAACTACATCAAGGAACTC | |
| | GATCGTATCATTGCATGCAAAAGAAAAAATCCCACATCCTGCT | |
| | CAAGACGCTTCTACCAGCTCACCAAGCTCCTGGACTCCGTGC | |
| | AGCCTATTGCGAGAGAGCTGCATCAGTTCACTTTTGACCTGCT | |
| | AATCAAGTCACACATGGTGAGCGTGGACTTTCCGGAAATGAT | |
| | GGCAGAGATCATCTCTGTGCAAGTGCCCAAGATCCTTTCTGG | |
| | GAAAGTCAAGCCCATCTATTTCCACACCCAGTGAAGGTAAGTC | |
| | TGGAGGCTTGCTTTGGGCTGTATGCTGAATACTTGAAGGGTA | |
| | GATTCATTTTGGCCTCTGACTGATGAATCTACTTTCAATATTCA | |
| | GGACACAAGGCCCTTATCAGCACTCTGGAGGCTTGCTTTGGG | |
| | CTGTATGCTGTTATAACAGGCAGAAGACATCTTTTGGCCTCTG | |
| | ACTGAGATGTCTCTTCTGTTATAACAGGACACAAGGCCCTTAT | |
| | CAGCACTCTGGAGGCTTGCTTTGGGCTGTATGCTGTTTAAAG | |
| | GCATACAACACCATTTTTGGCCTCTGACTGAATGGTGTGTTGC | |
| | CTTTAAACAGGACACAAGGCCCTTATCAGCACTCTCTCTCTCT | |
| | GCAGGGTATTTGTGAAATTTGTGATGCTATTGCTTTATTTGTAA | |
| | CCATCTAGCTTTATTTGTGAAATTTGTGATGCTATTGCTTTATTT | |
| | GTAACCATTATAAGCTGCAATAAACAAGTTAACAACAACAATTG | |
| | CATTCATTTTATGTTTCAGGTTCAGGGGGAGATGTGGGAGGTT | |
| | TTTTAAAGCGG | |
| AAV-0007 | TGGAGAACAGCCTGTTCTCCACACGTGTAATCCCGTGGGAGG | 46 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGATACTCTGGCTTCACAGTTTGGA | |
| | GACTGCCAGGGACCATGTTTTGCCCATTGACTATTACTTTCCA | |
| | CCCCAGAAGACCTGCCTGATCTGTGGAGATGAAGCTTCTGGG | |
| | TGTCACTATGGAGCTCTCACATGTGGAAGCTGCAAGGTCTTC | |
| | TTCAAAAGAGCCGCTGAAGGGAAACAGAAGTACCTGTGCGC | |
| | CAGCAGAAATGATTGCACTATTGATAAATTCCGAAGGAAAAATT | |
| | GTCCATCTTGTCGTCTTCGGAAATGTTATGAAGCAGGGATGAC | |
| | TCTGGGAGCCCGGAAGCTGAAGAAACTTGGTAATCTGAAACT | |
| | ACAGGAGGAAGGAGAGGCTTCCAGCACCACCAGCCCCACTG | |
| | AGGAGACAACCCAGAAGCTGACAGTGTCACACATTGAAGGCT | |
| | ATGAATGTCAGCCCATCTTTCTGAATGTCCTGGAAGCCATTGA | |
| | GCCAGGTGTAGTGTGTGCTGGACACGACAACAACCAGCCCG | |
| | ACTCCTTTGCAGCCTTGCTCTCTAGCCTCAATGAACTGGGAG | |
| | AGAGACAGCTTGTACACGTGGTCAAGTGGGCCAAGGCCTTG | |
| | CCTGGCTTCCGCAACTTACACGTGGACGACCAGATGGCTGTC | |
| | ATTCAGTACTCCTGGATGGGGCTCATGGTGTTTGCCATGGGC | |
| | TGGCGATCCTTCACCAATGTCAACTCCAGGATGCTCTACTTCG | |
| | CCCCTGATCTGGTTTTCAATGAGTACCGCATGCACAAGTCCC | |
| | GGATGTACAGCCAGTGTGTCCGAATGAGGCACCTCTCTCAAG | |
| | AGTTTGGATGGCTCCAAATCACCCCCCAGGAATTCCTGTGCA | |
| | TGAAAGCACTGCTACTCTTCAGCATTATTCCAGTGGATGGGCT | |
| | GAAAAATCAAAAATTCTTTGATGAACTTCGAATGAACTACATCA | |
| | AGGAACTCGATCGTATCATTGCATGCAAAAGAAAAAATCCCAC | |
| | ATCCTGCTCAAGACGCTTCTACCAGCTCACCAAGCTCCTGGA | |
| | CTCCGTGCAGCCTATTGCGAGAGAGCTGCATCAGTTCACTTT | |
| | TGACCTGCTAATCAAGTCACACATGGTGAGCGTGGACTTTCC | |
| | GGAAATGATGGCAGAGATCATCTCTGTGCAAGTGCCCAAGAT | |
| | CCTTTCTGGGAAAGTCAAGCCCATCTATTTCCACACCCAGTGA | |
| | AGGTAAGTCTGGAGGCTTGCTTTGGGCTGTATGCTGAATACTT | |
| | GAAGGGTAGATTCATTTTGGCCTCTGACTGATGAATCTACTTT | |
| | CAATATTCAGGACACAAGGCCCTTATCAGCACTCTGGAGGCTT | |
| | GCTTTGGGCTGTATGCTGTTATAACAGGCAGAAGACATCTTTT | |
| | GGCCTCTGACTGAGATGTCTCTTCTGTTATAACAGGACACAAG | |
| | GCCCTTATCAGCACTCTGGAGGCTTGCTTTGGGCTGTATGCT | |
| | GTTTAAAGGCATACAACACCATTTTTGGCCTCTGACTGAATGG | |
| | TGTGTTGCCTTTAAACAGGACACAAGGCCCTTATCAGCACTCT | |
| | CTCTCTCTGCAGGGTATTTGTGAAATTTGTGATGCTATTGCTTT | |
| | ATTTGTAACCATCTAGCTTTATTTGTGAAATTTGTGATGCTATTG | |
| | CTTTATTTGTAACCATTATAAGCTGCAATAAACAAGTTAACAACA | |
| | ACAATTGCATTCATTTTATGTTTCAGGTTCAGGGGGAGATGTG | |
| | GGAGGTTTTTTAAAGCGG | |
| AAV-0008 | TGGAGAACAGCCTGTTCTCCACACGTGTAATCCCGTGGGAGG | 47 |
| | TCAGACGTGCGTGTGGTAAAAGTATCTATGTTCAGAAAGGGAA | |
| | ATAGAAGCTGAATTAAGTAATTTTATACACACGTTATGGGGTGC | |
| | CCTTCTGTGAGTAATTCTTAAATTCAAAAGCAAATAGTTGTGAC | |
| | CACAAAGGGCTTAGTACATCTCAGCTGATTTTAGCTCTCAGAC | |
| | TCAAACTGGATATGATTGGTATTGACCTGCTCTGTGCATGTGAT | |
| | TTTAAGATGGTCTGACTCTTGTCTGTCATTCTGTTGGGTTATAT | |
| | TAATAACAGGGGACAAAGGGTGAAATAATCCAGTAAAGTTTTG | |
| | AGCAGCCACTTGACAACGTATTCCAAATAAATGAGAGGAGGA | |
| | AAACCCTAGCTCTACCAACTGGGGCCATAAAACAGAGTCTTCT | |
| | ACACTCTCTTTAATGTCAATATTAACCAGTTTGCAAATGTTCAG | |
| | TCAGTTATGGGCATGGATGGATGTCCTGAATGGGTTCCCGGG | |
| | ATAAAGTGTCATCATAGGACAGAAATCACAGGGAGAGTGCAC | |
| | CAAGGAAAAATTACAGTACTGCTATATTTACTTAGTGCCTCTGA | |
| | ACTAGGGTTTTATTTTCCACGGGTTGGAAAGGGAACCACCTG | |
| | TCTCAATTGCTGATGTCAGAGAGCTCCCTCGAGACACAGGGC | |
| | TGCTGGAAAGCACATGATACTGTACATATTTGCTCTTACGTTCG | |
| | TATCTGGCTAAGATTGGGTTTCAGATTTGTGCCCTATTGTGGA | |
| | GTTCATTTAGTAGTGACTCTGAGATGCCCTCCCACGTCACCAT | |
| | GCCCTTGGAGGTCAGACGTGCGTGTGCACGTGTAATCCCGT | |
| | GACCGGTGCCGCCACCATGGAAGTGCAGTTAGGGCTGGGAA | |
| | GGGTCTACCCTCGGCCGCCGTCCAAGACCTACCGAGGAGCT | |
| | TTCCAGAATCTGTTCCAGAGCGTGCGCGAAGTGATCCAGAAC | |
| | CCGGGCCCCAGGCACCCAGAGGCCGCGAGCGCAGCACCTC | |
| | CCGGCGCCAGTTTGCTGCTGCTGCAGCAGCAGCAGCAGCAG | |
| | CAGCAGCAGCAGCAGCAGCAGCAGCAGCAGCAGCAGCAGC | |
| | AGCAGCAGCAAGAGACTAGCCCCAGGCAGCAGCAGCAGCAG | |
| | CAGGGTGAGGATGGTTCTCCCCAAGCCCATCGTAGAGGCCC | |
| | CACAGGCTACCTGGTCCTGGATGAGGAACAGCAACCTTCACA | |
| | GCCGCAGTCGGCCCTGGAGTGCCACCCCGAGAGAGGTTGC | |
| | GTCCCAGAGCCTGGAGCCGCCGTGGCCGCCAGCAAGGGGC | |
| | TGCCGCAGCAGCTGCCAGCACCTCCGGACGAGGATGACTCA | |
| | GCTGCCCCATCCACGTTGTCCCTGCTGGGCCCCACTTTCCCC | |
| | GGCTTAAGCAGCTGCTCCGCTGACCTTAAAGACATCCTGAGC | |
| | GAGGCCAGCACCATGCAACTCCTTCAGCAACAGCAGCAGGA | |
| | AGCAGTATCCGAAGGCAGCAGCAGCGGGAGAGCGAGGGAG | |
| | GCCTCGGGGGCTCCCACTTCCTCCAAGGACAATTACTTAGGG | |
| | GGCACTTCGACCATTTCTGACAACGCCAAGGAGTTGTGTAAG | |
| | GCAGTGTCGGTGTCCATGGGCCTGGGTGTGGAGGCGTTGGA | |
| | GCATCTGAGTCCAGGGGAACAGCTTCGGGGGGATTGCATGTA | |
| | CGCCCCACTTTTGGGAGTTCCACCCGCTGTGCGTCCCACTC | |
| | CTTGTGCCCCATTGGCCGAATGCAAAGGTTCTCTGCTAGACG | |
| | ACAGCGCAGGCAAGAGCACTGAAGATACTGCTGAGTATTCCC | |
| | CTTTCAAGGGAGGTTACACCAAAGGGCTAGAAGGCGAGAGC | |
| | CTAGGCTGCTCTGGCAGCGCTGCAGCAGGGAGCTCCGGGAC | |
| | ACTTGAACTGCCGTCTACCCTGTCTCTCTACAAGTCCGGAGC | |
| | ACTGGACGAGGCAGCTGCGTACCAGAGTCGCGACTACTACAA | |
| | CTTTCCACTGGCTCTGGCCGGACCGCCGCCCCCTCCGCCGC | |
| | CTCCCCATCCCCACGCTCGCATCAAGCTGGAGAACCCGCTG | |
| | GACTACGGCAGCGCCTGGGCGGCTGCGGCGGCGCAGTGCC | |
| | GCTATGGGGACCTGGCGAGCCTGCATGGCGCGGGTGCAGC | |
| | GGGACCCGGTTCTGGGTCACCCTCAGCCGCCGCTTCCTCAT | |
| | CCTGGCACACTCTCTTCACAGCCGAAGAAGGCCAGTTGTATG | |
| | GACCGTGTGGTGGTGGTGGGGGTGGTGGCGGCGGCGGCG | |
| | GCGGCGGCGGCGGCGGCGGCGGCGGCGGCGGCGGCGGC | |
| | GAGGCGGGAGCTGTAGCCCCCTACGGCTACACTCGGCCCCC | |
| | TCAGGGGCTGGCGGGCCAGGAAAGCGACTTCACCGCACCTG | |
| | ATGTGTGGTACCCTGGCGGCATGGTGAGCAGAGTGCCCTATC | |
| | CCAGTCCCACTTGTGTCAAAAGCGAAATGGGCCCCTGGATGG | |
| | ATAGCTACTCCGGACCTTACGGGGACATGCGTTTGGAGACTG | |
| | CCAGGGACCATGTTTTGCCCATTGACTATTACTTTCCACCCCA | |
| | GAAGACCTGCCTGATCTGTGGAGATGAAGCTTCTGGGTGTCA | |
| | CTATGGAGCTCTCACATGTGGAAGCTGCAAGGTCTTCTTCAAA | |
| | AGAGCCGCTGAAGGGAAACAGAAGTACCTGTGCGCCAGCAG | |
| | AAATGATTGCACTATTGATAAATTCCGAAGGAAAAATTGTCCAT | |
| | CTTGTCGTCTTCGGAAATGTTATGAAGCAGGGATGACTCTGG | |
| | GAGAAAAATTCCGGGTTGGCAATTGCAAGCATCTCAAAATGAC | |
| | CAGACCCTGAAGGTAAGTCTGGAGGCTTGCTTTGGGCTGTAT | |
| | GCTGAATACTTGAAGGGTAGATTCATTTTGGCCTCTGACTGAT | |
| | GAATCTACTTTCAATATTCAGGACACAAGGCCCTTATCAGCAC | |
| | TCTGGAGGCTTGCTTTGGGCTGTATGCTGTTATAACAGGCAGA | |
| | AGACATCTTTTGGCCTCTGACTGAGATGTCTCTTCTGTTATAAC | |
| | AGGACACAAGGCCCTTATCAGCACTCTGGAGGCTTGCTTTGG | |
| | GCTGTATGCTGTTTAAAGGCATACAACACCATTTTTGGCCTCT | |
| | GACTGAATGGTGTGTTGCCTTTAAACAGGACACAAGGCCCTT | |
| | ATCAGCACTCTCTCTCTCTGCAGGGTATTTGTGAAATTTGTGA | |
| | TGCTATTGCTTTATTTGTAACCATCTAGCTTTATTTGTGAAATTT | |
| | GTGATGCTATTGCTTTATTTGTAACCATTATAAGCTGCAATAAAC | |
| | AAGTTAACAACAACAATTGCATTCATTTTATGTTTCAGGTTCAG | |
| | GGGGAGATGTGGGAGGTTTTTTAAAGCGG | |

Western blot of lysates from muscle tissue of NHPs infected with the AAV-0001 (animal #19081609) or the AAV-0004 (animal #1606020) were analyzed for the androgen receptor compared to PBS control (animal #16042307). FIG. 3B is a schematic diagram illustrating the western blot of lysates from the muscle tissue of NHPs infected with the AAV-0001 and the AAV-0004 according to the present disclosure. As shown in FIG. 3B, The expression of endogenous androgen receptor in the animal #16042307 was readily detected at around 110 kDa. The expression of endogenous androgen receptor was replaced in the animal #1606020 infected with the AAV-0004 encoding a mutant form of the human androgen receptor having an excess number of glutamine in its N-terminus rendering the protein larger in size. The expression of endogenous androgen receptor was replaced in the animal #19081609 infected with the AAV-0001 encoding a wild type form of the human androgen receptor which is slightly larger in size compared to the endogenous monkey androgen receptor.

### Example 4: Therapeutic Potential of AAV-0001 in SBMA Patient

Induced polypotent stem cells (iPSCs) were derived from an SBMA patient (SBMA-iPSC) and his normal sibling (Control-iPSC). The iPSCs were induced to differentiate into motor neurons and expression level of a marker gene natriuretic peptide B (NPPB) were compared by the qRT-PCR. The iPSCs were induced to differentiate into motor neurons, and the expression level of the marker gene natriuretic peptide B (NPPB) was compared by qRT-PCR.

NPPB, a hormone primarily involved in regulating cardiovascular function, has been identified as a key marker gene in various neurodegenerative diseases, including SBMA. In the context of SBMA, a neurodegenerative disorder caused by mutations in the androgen receptor gene, the expression of NPPB has shown to be upregulated as a compensatory response to cellular stress and neuronal injury. SBMA leads to the progressive degeneration of motor neurons, and as part of the cellular response, increased NPPB expression may indicate an attempt to restore or protect motor neuron function. This makes NPPB an ideal candidate as a marker gene for SBMA.

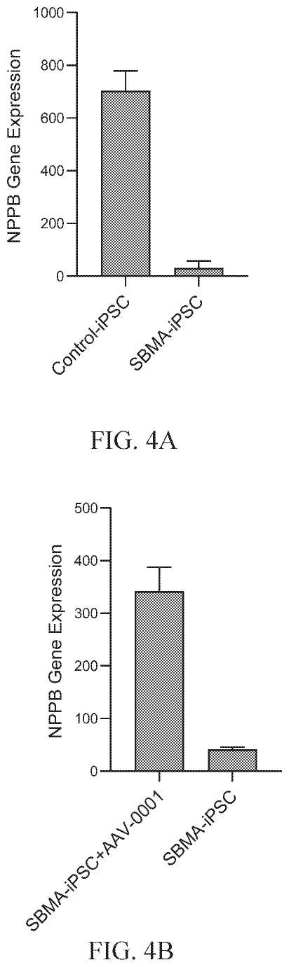

FIG. 4A is a schematic diagram illustrating NPPB gene expressions of the iPSCs according to the present disclosure. The expression of NPPB was significantly reduced in the SBMA-iPSC motor neurons than normal control.

SBMA-iPSCs (2.5×10⁵ cells/well) were infected with the AAV-0001 at 1.0×10¹² genome copies per well for 2 days and then differentiated into motor neurons and further cultured for another 28 days. Expression of marker gene NPPB was then assessed by the qRT-PCR. FIG. 4B is a schematic diagram illustrating NPPB gene expressions of the SBMA-iPSCs infected with the AAV-0001 according to the present disclosure. As shown in FIG. 4B, The AAV-0001 significantly enhanced the expression level of marker gene NPPB indicative of partial restoration of the defective function of androgen receptor in SBMA, suggesting a therapeutic potential of the AAV-0001 in mitigating the loss of motor neuron function in the SBMA patient.

The iPSCs were induced to differentiate into motor neurons and expression level of a marker gene glycoprotein nonmetastatic melanoma protein B (GPNMB) were compared by qRT-PCR.

GPNMB has been associated with the activation of glial cells, such as microglia and astrocytes, which play a role in the inflammatory response to neuronal injury. SBMA is characterized by motor neuron degeneration due to mutations in the androgen receptor gene. The upregulation of GPNMB in neuroinflammation is believed to have neuroprotective functions to some extent. This neurorepair effect makes the upregulation of GPNMB a potential positive marker in the process of neuronal damage repair.

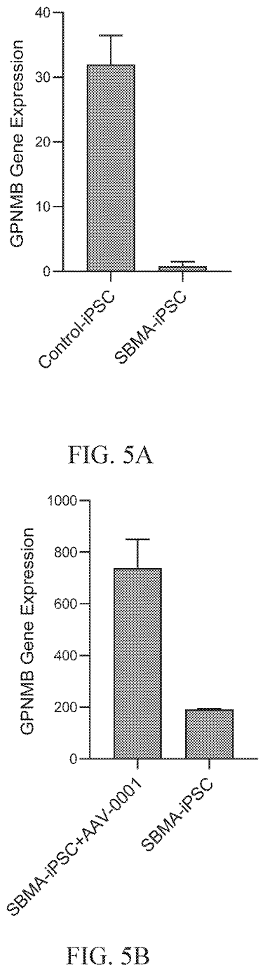

FIG. 5A is a schematic diagram illustrating GPNMB gene expressions of the iPSCs according to the present disclosure. The expression of GPNMB was significantly reduced in SBMA-iPSC motor neurons than normal control.

Expression of marker gene GPNMB was then assessed by the qRT-PCR. FIG. 5B is a schematic diagram illustrating GPNMB gene expressions of the SBMA-iPSCs were infected with the AAV-0001 according to the present disclosure. The AAV-0001 significantly enhanced the expression level of marker gene GPNMB indicative of partial restoration of the defective function of androgen receptor in SBMA, suggesting a therapeutic potential of the AAV-0001 in mitigating the loss of motor neuron function in the SBMA patient.

The iPSCs were induced to differentiate into motor neurons and expression level of a marker gene phosphodiesterase 7B (PDE7B) were compared by the qRT-PCR.

PDE7B is an enzyme involved in the regulation of cyclic adenosine monophosphate (cAMP) signaling, playing a role in various cellular processes, including those related to inflammation and neuronal function. Inhibition of PDE7B has shown promise for treating neurodegenerative diseases, as it can modulate the inflammatory response and promote neuroprotection by regulating cAMP signaling. Given its role in neuroinflammation and neuronal survival, the expression level of PDE7B can serve as an important indicator of the disease's activity and the degree of neuroinflammation. Therefore, PDE7B can be considered a valuable marker gene for SBMA.

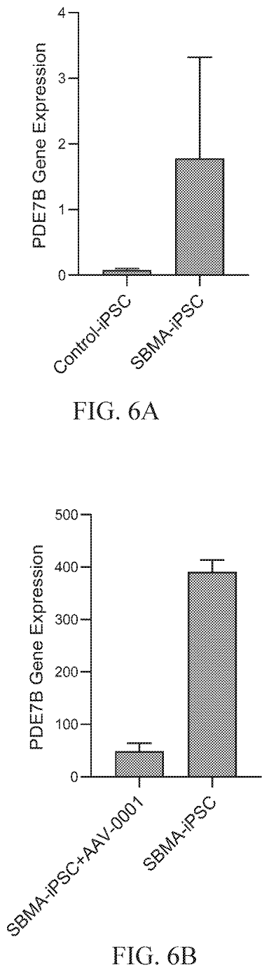

FIG. 6A is a schematic diagram illustrating PDE7B gene expressions of the iPSCs according to the present disclosure. The expression of PDE7B was significantly elevated in SBMA-iPSC motor neurons than normal control.

Expression of marker gene PDE7B was then assessed by the qRT-PCR. FIG. 6B is a schematic diagram illustrating PDE7B gene expressions of the SBMA-iPSCs were infected with the AAV-0001 according to the present disclosure. The AAV-0001 significantly suppressed the expression level of marker gene PDE7B indicative of alleviation of the toxic gain-of-function due to the Poly-Q expanded androgen receptor in SBMA, suggesting a therapeutic potential of the AAV-0001 in mitigating the loss of motor neuron function in the SBMA patient.

The iPSCs were induced to differentiate into motor neurons and expression level of a marker gene sonic hedgehog signaling molecule (SHH) were compared by the qRT-PCR.

SHH pathway affects neurogenesis and neural patterning during the development of the central nervous system. Upregulation of the marker gene SHH typically indicates a response to neurodegenerative processes, particularly in relation to motor neuron dysfunction.

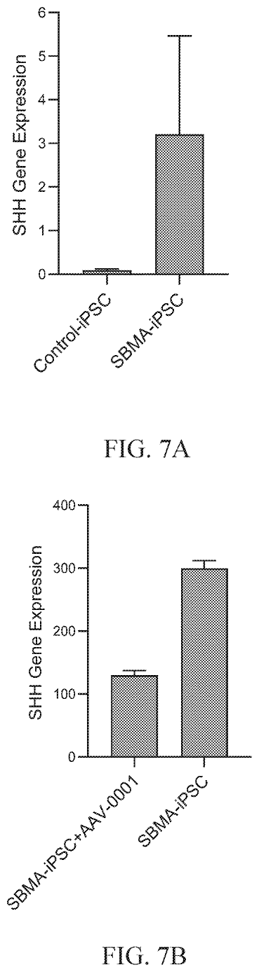

FIG. 7A is a schematic diagram illustrating SHH gene expressions of the iPSCs according to the present disclosure. The expression of SHH was significantly elevated in SBMA-iPSC motor neurons than normal control.

Expression of marker gene SHH was then assessed by the qRT-PCR. FIG. 7B is a schematic diagram illustrating SHH gene expressions of the SBMA-iPSCs were infected with the AAV-0001 according to the present disclosure. The AAV-0001 significantly suppressed the expression level of marker gene SHH indicative of alleviation of the toxic gain-of-function due to the Poly-Q expanded androgen receptor in SBMA, suggesting a therapeutic potential of the AAV-0001 in mitigating the loss of motor neuron function in the SBMA patient.

What is claimed is:

1. A nucleic acid molecule comprising a promoter module, an expression module, and a regulatory module, wherein:
the promoter module is configured to control transcription of the expression module and/or the regulatory module,
the expression module includes a first gene of interest (GOI), which encodes a first target protein that is an exogenous protein to a cell,
the regulatory module includes one or more nucleotide segments that are configured to regulate a second GOI and/or a second target protein encoded by the second GOI, wherein the second target protein is an endogenous protein of the cell,
the first target protein at least partially and functionally substitutes the second target protein in the cell, or attenuates the second target protein in the cell, and
the first GOI and the second GOI are not identical but have a sequence identity of at least 80%, 85%, 90%, 95%, or 98%.

2. The nucleic acid molecule of claim 1, wherein the first target protein is an androgen receptor (AR) that includes a polyglutamine (poly-Q) segment with less than 32 glutamines.

3. The nucleic acid molecule of claim 1, wherein the first target protein has one of amino acid sequences of SEQ ID NOs: 1, 36, or 38.

4. The nucleic acid molecule of claim 1, wherein the first GOI is an AR gene or one of its variants.

5. The nucleic acid molecule of claim 1, wherein the first GOI has one of DNA sequences of SEQ ID NOs: 2, 37, or 39.

6. The nucleic acid molecule of claim 1, wherein the second target protein is an AR that causes spinal-bulbar muscular atrophy (SBMA) and includes a polyglutamine (poly-Q) segment with at least 32 glutamines.

7. The nucleic acid molecule of claim 1, wherein the second target protein has an amino acid sequence of SEQ ID NO: 3, or the second GOI has a DNA sequence of SEQ ID NO: 4.

8. The nucleic acid molecule of claim 1, wherein the regulatory module is configured to down-regulate an mRNA encoded by the second GO by targeting 5′-UTR or 3′-UTR of the mRNA.

9. The nucleic acid molecule of claim 1, wherein the each of one or more nucleotide segments is configured to encode one or more double stranded ribonucleic acid (dsRNA) agents, and each of the one or more double stranded ribonucleic acid (dsRNA) agents includes a sense strand and an anti-sense strand.

10. The nucleic acid molecule of claim 9, wherein one of the one or more nucleotide segments encode an RNA sequence of SEQ ID NOs: 5, 6, 7, or 8.

11. The nucleic acid molecule of claim 1, wherein the promoter module includes a promoter that controls transcription of the expression module and the regulatory module and a physiological signal responsive promoter sequence, and wherein the promoter module responds to a physiological signal.

12. The nucleic acid molecule of claim 11, wherein the promoter module includes an Androgen Response Element (ARE) sequence and a Kozak sequence.

13. The nucleic acid molecule of claim 1, wherein the nucleic acid molecule is a DNA molecule, and in a direction from 5′ to 3′, comprises a nucleotide sequence as set forth in formula (I): 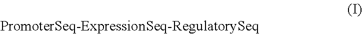
wherein PromoterSeq represents a nucleotide sequence of the promoter module;
ExpressionSeq represents a nucleotide sequence of the expression module; and
RegulatorySeq represents a nucleotide sequence of the regulatory module.

14. The nucleic acid molecule of claim 13, wherein the PromoterSeq has a nucleotide sequence that has at least 80%, 85%, 90%, 95%, 98%, or 99% identity to SEQ ID NO: 48.

15. The nucleic acid molecule of claim 13, having a nucleotide sequence that has at least 80%, 85%, 90%, 95%, 98%, or 99% identity to any one of SEQ ID NOs: 40-47.

16. The nucleic acid molecule of claim 13, having a nucleotide sequence of any one of SEQ ID NOs: 40-47.

17. A double stranded ribonucleic acid (dsRNA) agent for inhibiting expression of androgen receptor (AR), comprising a sense strand and an antisense strand, wherein
the sense strand comprises at least 21 contiguous nucleotides differing by no more than 5 nucleotides from any one of SEQ ID NOs: 13, 15, 17, 19, 21, 23, 25, 27, and 29, and the antisense strand comprises a nucleotide sequence that is at least partially complementary to the sense strand.

18. The dsRNA agent of claim 17, wherein the sense strand has a nucleotide sequence of SEQ ID NO: 13, 15, 17, 19, 21, 23, 25, 27, or 29 and the anti-sense RNA strand has a nucleotide sequence of SEQ ID NO: 14, 16, 18, 20, 22, 24, 26, 28, or 30.

19. A pharmaceutical composition comprising the nucleic acid molecule of claim 1.

20. A method for treating a subject suffering from SBMA, comprising administering the nucleic acid molecule of claim 1 to the subject.
